SEQUENCE LISTING (92 sequences)

SEQ ID NO: 1 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISRINDIVLELKGSETTFSCEYADETATIVEFLNRWITFAQSIISTLT

SEQ ID NO: 3 - protein, 366 aa, synthetic construct
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 4 - protein, 366 aa, synthetic construct
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISRINDIVLELKGSETTFSCEYADETATIVEFLNRWITFAQSIISTLT

SEQ ID NO: 5 - DNA, 1098 bp, synthetic construct
  source 1..1098: mol_type = other DNA
caagtgcagctcgtgcagtccggtggcggacctgtcaagcctgggggatcactgagactgtcgtgcgccgcctccgggtttatgttccgggcttattcgatgaactgggtcagacaggcgccgggaaagggcctggaatgggtgtcaagcatttcctcgtccggacgctacatccactacgccgattccgtgaagggtcgcttcaccatttctcgggacaacgccaagaattccctctacctccaaatgaactcccttcgggccgaggacaccgccgtgtactactgcgctcgggagactgtgatggccggaaaggccctggactactggggacagggaaccctcgtgactgtgtcctccgcatcaactaagggccccagcgtgttcccactggcccctagctccaagagcaccagcgaaggaaccgcagcactgggttgtcttgtgaaggactacttcccggagcccgtgacagtgtcctggaactccggggccctgactagcggagtccacactttccccgcggtgctgcagtcgtccggattgtacagcctgtccagtgtcgtgaccgtgcctagctcgagcctgggtactcagacttacatctgcaacgtcaaccacaagccctccaacaccaaagtggacaagaaagtcgagccgaagtcatccggcggcggcggaagcggcgggggcggctcggcgcccacctcctcatccaccaagaaaacccagctgcagttggagcatctgctgctggatctccaaatgattcttaatgggatcaacaactataagaacccaaagctgactcggatgctgaccttcaagttctacatgccgaagaaagccaccgagctcaagcatctccagtgcttggaagaagaactgaagccgctggaagaggtgctgaacctggcccagtccaagaactttcacctccggccgagggatctgattagccgcatcaacgacatcgtcctcgagctgaagggctcggaaaccaccttttcctgcgaatacgcggacgaaaccgccactatcgtggaattcctgaaccgctggatcaccttcgctcaatcgatcatctccacgctgacc

SEQ ID NO: 6 - protein, 223 aa, synthetic construct
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 7 - DNA, 669 bp, synthetic construct
  source 1..669: mol_type = other DNA
caggtgcagcttgtccagtccggaggcggccctgtgaagcctggggggtcattgagattgtcctgtgcagcatccggatttatgttcagggcttactccatgaactgggtgcgccaggctccagggaaggggttggaatgggtgtccagcatcagcagctctggaaggtacatccactacgccgactccgtgaagggcaggttcaccatttcccgggacaacgctaagaacagcctgtacctccagatgaactccctccgggctgaggacaccgccgtgtactactgcgcccgggagacagtgatggccgggaaagccctggattactggggccaaggcactctggtcacagtcagctcagcctctacaaaaggccctagtgtgtttcctctggccccatcaagtaagagcacctcagaggggaccgccgccctgggctgcctggtcaaagattactttccagagcccgtgacagtgagttggaacagtggggctctgacaagcggggtccatacatttcccgccgtgctgcagagcagcgggctgtatagcctgagcagcgtcgtcactgtgcccagctcttctctcggcacacagacttacatttgcaatgtgaaccacaagcccagcaacactaaggtcgataagaaggtggagcccaagtcctcc

SEQ ID NO: 8 - DNA, 669 bp, synthetic construct
  source 1..669: mol_type = other DNA
caagtgcaactggttcaatctggcggcggccccgtaaagccaggtggtagcctgcgtctgagttgtgccgctagtgggttcatgttccgagcctactctatgaattgggtgaggcaggccccagggaaaggtttggagtgggtttcctcaatcagctcaagtggtcgatatattcactatgctgactcagtcaagggtcgtttcaccatcagtcgggataatgccaagaacagcttgtatcttcaaatgaacagcttgagagctgaagataccgctgtgtactattgtgctagggaaactgtcatggctggcaaggccttggactactgggggcagggtactcttgtaactgttagtagcgcaagcactaaaggcccatcagtttttccattggcaccatcatccaaatctacatctgagggcactgcagcattggggtgtctcgtcaaagattattttcccgagccagtcacagtatcctggaatagcggggctttgacatctggggtgcatacatttcctgctgttttgcaatccagcggactctactctctgagttctgttgtaacagtccctagcagttctctgggaacccaaacttacatttgtaacgtgaatcacaagccttccaacacaaaggtcgataaaaaggtcgagcctaaatcatcc

SEQ ID NO: 9 - protein, 364 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFAQSIISTLT

SEQ ID NO: 10 - DNA, 60 bp, synthetic construct
  source 1..60: mol_type = other DNA
atggagacccacagccaggtgttcgtgtacatgctgctgtggctgagcggggtggagggg

SEQ ID NO: 11 - DNA, 669 bp, synthetic construct
  misc_feature 1..669: note = SL335H
  source 1..669: mol_type = other DNA
caagtgcaattggtgcagtcaggtggcggcccagtaaaacccggtgggtctctgagattgagctgcgctgcatccggatttatgttccgtgcctacagcatgaattgggtgcggcaagctcctggaaagggactcgaatgggtttccagtattagcagttctgggaggtatatacattatgctgactcagtgaaagggaggttcacaattagccgggacaatgccaaaaactctctctatctgcagatgaacagccttcgcgccgaagataccgccgtttattactgcgctcgggagactgtgatggccggtaaggctcttgactattggggacagggaactttggtgactgtttcatctgcctctacaaagggacccagcgtttttccattggcacctagtagcaagtccacatctgaaggtacagctgctttggggtgtttggtgaaagactacttccccgaaccagtgacagtttcatggaactccggcgctttgactagtggagtgcataccttcccagctgttcttcagagtagtgggctttatagtttgagtagcgtcgtgactgtcccatcatcctctctcggcacacaaacctatatctgcaatgtaaaccataagccatcaaacaccaaagtagacaagaaagttgagccaaagtccagc

SEQ ID NO: 12 - DNA, 669 bp, synthetic construct
  misc_feature 1..669: note = SL335H
  source 1..669: mol_type = other DNA
caagtccagcttgtgcagtccggcggtggaccagtgaagcctgggggatcactgcgcctctcctgtgccgcttcggggttcatgttccgggcatactcgatgaactgggttagacaggctcccggaaagggcctggaatgggtgtccagtatctcaagctcgggccgctacattcattatgcggacagcgtgaagggcagattcaccattagccgggacaatgccaagaactccctgtacttgcaaatgaactccctgagggccgaggatactgcggtgtactattgtgctcgggagactgtgatggccggaaaggccctggactactggggccagggcacccttgtgaccgtgtcctccgcctcgaccaagggaccgtcggtgtttcctctggcgccgtcctcgaagtcaacctccgagggaaccgccgccctgggttgcctcgtgaaagactacttccctgaacccgtgactgtgtcctggaacagcggtgccctgacctccggggtgcatactttccctgcggtgctgcagtcgtccggactctactccctgtcatccgtcgtgaccgtgccttcctcctccctgggaacccagacctacatctgcaacgtgaaccacaagccctccaacaccaaggtcgacaagaaagtcgagcccaagtccagc

SEQ ID NO: 13 - protein, 215 aa, synthetic construct
  REGION 1..215: note = SL335L
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 14 - DNA, 645 bp, synthetic construct
  misc_feature 1..645: note = SL335L
  source 1..645: mol_type = other DNA
gacatcgtcctgacccagagccccgggacactgagcctgtcccccggggagacagccacactgagctgcagggccagccagtccgtcggctccaacctggcctggtaccagcagaagcccgggcaggcccccaggctgctgatttacggcgccagcacaggcgccacaggggtgcccgccaggtttagcggcagccggagcggcacagattttacactgacaatcacatccctgcagcccgaggattttgccacatactactgccagcagtactactccttcctggccaagacattcggccagggcacccagctggagatcaagcggaccgtggccgcccccagcgtgtttattttcccccccagcgatgagcagctgaagtccggcaccgcctccgtcgtgtgcctgctgaacaacttctacccccgggaggccaaggtccagtggaaggtcgataacgccctgcagagcgggaactcccaggagtccgtgaccgagcaggactccaaggacagcacctactccctgtccaacaccctgaccctgagcaaggccgactacgagaagcacaaggtctacgcctgcgaggtgacccaccagggcctgtcctcccccgtgaccaagagctttaaccggggggagtcc

SEQ ID NO: 15 - DNA, 645 bp, synthetic construct
  misc_feature 1..645: note = SL335L
  source 1..645: mol_type = other DNA
gatatcgtgctgactcagtcgcctggtaccctttccctgtcacccggagaaactgccaccctgagctgtagagcgagccagtccgtgggctccaacctggcctggtatcaacagaagcctgggcaggcccctcgcctgttgatctacggcgccagcactggtgccaccggcgtgccagctcggttctccggctcccggtcggggactgacttcaccctcaccattacgagcctgcagcccgaagatttcgcgacctactactgccaacagtactactcattcctggctaagactttcggacaaggcacccagctcgagatcaagcggaccgtggcagcaccgtccgtgttcattttcccgccgtccgacgagcaacttaagtccggaaccgcctctgtcgtgtgcctcctcaacaacttctacccgcgcgaggccaaggtccagtggaaggtcgacaacgcgctgcagtccggaaattcacaggaaagcgtgaccgaacaggactccaaggattcgacctactccctgtccaacactctgaccctgtcgaaagccgactacgagaagcacaaagtgtacgcctgcgaagtgacacatcagggactcagctcgcccgtgaccaagagcttcaacaggggagagtcg

SEQ ID NO: 16 - protein, 8 aa, synthetic construct
  REGION 1..8: note = Linker 1
GSAPAPGS

SEQ ID NO: 17 - DNA, 24 bp, synthetic construct
  misc_feature 1..24: note = Linker 1
  source 1..24: mol_type = other DNA
gggtcagcccctgccccaggtagc

SEQ ID NO: 18 - DNA, 24 bp, synthetic construct
  misc_feature 1..24: note = Linker 1
  source 1..24: mol_type = other DNA
ggctcggccccggcaccgggcagc

SEQ ID NO: 19 - protein, 19 aa, synthetic construct
MEWSWVFLFFLSVTTGVLS

SEQ ID NO: 20 - DNA, 57 bp, synthetic construct
  source 1..57: mol_type = other DNA
atggagtggtcctgggtcttcctcttcttcctcagcgtgaccaccggagtgctttcc

SEQ ID NO: 21 - protein, 20 aa, unidentified
METHSQVFVYMLLWLSGVEG

SEQ ID NO: 22 - protein, 5 aa, synthetic construct
  REGION 1..5: note = Heavy chain variable domain CDR1
SYGIS

SEQ ID NO: 23 - protein, 17 aa, synthetic construct
  REGION 1..17: note = Heavy chain variable domain CDR2
WINTYSGGTKYAQKFQG

SEQ ID NO: 24 - protein, 15 aa, synthetic construct
  REGION 1..15: note = Heavy chain variable domain CDR3
LGHCQRGICSDALDT

SEQ ID NO: 25 - protein, 17 aa, synthetic construct
  REGION 1..17: note = Heavy chain variable domain CDR2
RINTYNGNTGYAQRLQG

SEQ ID NO: 26 - protein, 5 aa, synthetic construct
  REGION 1..5: note = Heavy chain variable domain CDR1
NYGIH

SEQ ID NO: 27 - protein, 17 aa, synthetic construct
  REGION 1..17: note = Heavy chain variable domain CDR2
SISYDGSNKYYADSVKG

SEQ ID NO: 28 - protein, 16 aa, synthetic construct
  REGION 1..16: note = Heavy chain variable domain CDR3
DVHYYGSGSYYNAFDI

SEQ ID NO: 29 - protein, 5 aa, synthetic construct
  REGION 1..5: note = Heavy chain variable domain CDR1
SYAMS

SEQ ID NO: 30 - protein, 17 aa, synthetic construct
  REGION 1..17: note = Heavy chain variable domain CDR2
VISHDGGFQYYADSVKG

SEQ ID NO: 31 - protein, 11 aa, synthetic construct
  REGION 1..11: note = Heavy chain variable domain CDR3
AGWLRQYGMDV

SEQ ID NO: 32 - protein, 5 aa, synthetic construct
  REGION 1..5: note = Heavy chain variable domain CDR1
AYWIA

SEQ ID NO: 33 - protein, 17 aa, synthetic construct
  REGION 1..17: note = Heavy chain variable domain CDR2
MIWPPDADARYSPSFQG

SEQ ID NO: 34 - protein, 8 aa, synthetic construct
  REGION 1..8: note = Heavy chain variable domain CDR3
LYSGSYSP

SEQ ID NO: 35 - protein, 5 aa, synthetic construct
  REGION 1..5: note = Heavy chain variable domain CDR1
AYSMN

SEQ ID NO: 36 - protein, 17 aa, synthetic construct
  REGION 1..17: note = Heavy chain variable domain CDR2
SISSSGRYIHYADSVKG

SEQ ID NO: 37 - protein, 11 aa, synthetic construct
  REGION 1..11: note = Heavy chain variable domain CDR3
ETVMAGKALDY

SEQ ID NO: 38 - protein, 11 aa, synthetic construct
  REGION 1..11: note = Light chain variable domain CDR1
RASQSISRYLN

SEQ ID NO: 39 - protein, 7 aa, synthetic construct
  REGION 1..7: note = Light chain variable domain CDR2
GASRLES

SEQ ID NO: 40 - protein, 9 aa, synthetic construct
  REGION 1..9: note = Light chain variable domain CDR3
QQSDSVPVT

SEQ ID NO: 41 - protein, 11 aa, synthetic construct
  REGION 1..11: note = Light chain variable domain CDR1
RASQSISSYLN

SEQ ID NO: 42 - protein, 7 aa, synthetic construct
  REGION 1..7: note = Light chain variable domain CDR2
AASSLQS

SEQ ID NO: 43 - protein, 10 aa, synthetic construct
  REGION 1..10: note = Light chain variable domain CDR3
QQSYSTPPYT

SEQ ID NO: 44 - protein, 11 aa, synthetic construct
  REGION 1..11: note = Light chain variable domain CDR1
RASQSIFNYVA

SEQ ID NO: 45 - protein, 7 aa, synthetic construct
  REGION 1..7: note = Light chain variable domain CDR2
DASNRAT

SEQ ID NO: 46 - protein, 11 aa, synthetic construct
  REGION 1..11: note = Light chain variable domain CDR3
QQRSKWPPTWT

SEQ ID NO: 47 - protein, 12 aa, synthetic construct
  REGION 1..12: note = Light chain variable domain CDR1
RASETVSSRQLA

SEQ ID NO: 48 - protein, 7 aa, synthetic construct
  REGION 1..7: note = Light chain variable domain CDR2
GASSRAT

SEQ ID NO: 49 - protein, 9 aa, synthetic construct
  REGION 1..9: note = Light chain variable domain CDR3
QQYGSSPRT

SEQ ID NO: 50 - protein, 12 aa, synthetic construct
  REGION 1..12: note = Light chain variable domain CDR1
RASQSVSSSSLA

SEQ ID NO: 51 - protein, 9 aa, synthetic construct
  REGION 1..9: note = Light chain variable domain CDR3
QKYSSYPLT

SEQ ID NO: 52 - protein, 11 aa, synthetic construct
  REGION 1..11: note = Light chain variable domain CDR1
RASQSVGSNLA

SEQ ID NO: 53 - protein, 7 aa, synthetic construct
  REGION 1..7: note = Light chain variable domain CDR2
GASTGAT

SEQ ID NO: 54 - protein, 10 aa, synthetic construct
  REGION 1..10: note = Light chain variable domain CDR3
QQYYSFLAKT

SEQ ID NO: 55 - protein, 124 aa, synthetic construct
  REGION 1..124: note = Heavy chain variable domain 1
QVQLLQSGAEVKKPGASVKVSCKASGYTFTSYGISWVRQAPGQGLEWVGWINTYSGGTKYAQKFQGRVTMTRDTSISTVYMELSGLKSDDTAVYYCARLGHCQRGICSDALDTWGQGTLVTVSS

SEQ ID NO: 56 - protein, 124 aa, synthetic construct
  REGION 1..124: note = Heavy chain variable domain 2
EVQLLQSGAEVKEPGASVKVSCKASGYTFSSYGISWVRQAPGQGLEWVGRINTYNGNTGYAQRLQGRVTMTTDTSTSIAYMEVRSLRSDDTAVYYCARLGHCQRGICSDALDTWGQGTMVTVSS

SEQ ID NO: 57 - protein, 125 aa, synthetic construct
  REGION 1..125: note = Heavy chain variable domain 3
QVQLVQSGGGVVQTGGSLRLSCAASGFTFRNYGIHWVRQAPGKGLEWVASISYDGSNKYYADSVKGRFTISRDNSRNTVHVQMDSLRGGDTAVYYCARDVHYYGSGSYYNAFDIWGQGTLVTVSS

SEQ ID NO: 58 - protein, 120 aa, synthetic construct
  REGION 1..120: note = Heavy chain variable domain 4
QVQLVQSGGGLVQPGGSLRLSCAASGFTFSSYAMSWVRQAPGKGLEWLSVISHDGGFQYYADSVKGRFTVSRDNSKNTLYLQMNSLRAEDTAVYYCARAGWLRQYGMDVWGQGTLVTVSS

SEQ ID NO: 59 - protein, 117 aa, synthetic construct
  REGION 1..117: note = Heavy chain variable domain 5
EVQLVQSGTEVKKPGESLKISCKISGYSFTAYWIAWVRQMPGKGLEWMGMIWPPDADARYSPSFQGQVTFSVDKSISTAYLQWHSLKTSDTAVYYCARLYSGSYSPWGQGTLVTVSS

SEQ ID NO: 60 - protein, 120 aa, synthetic construct
  REGION 1..120: note = Heavy chain variable domain 6
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSS

SEQ ID NO: 61 - protein, 108 aa, synthetic construct
  REGION 1..108: note = Light chain variable domain 1
ELVLTQSPSSLSASVGDRVTITCRASQSISRYLNWYQQKPGKAPKLLIYGASRLESGVPSRFSGSGSGTDFTLTISSLQPEDFATYYCQQSDSVPVTFGQGTRLEIKR

SEQ ID NO: 62 - protein, 109 aa, synthetic construct
  REGION 1..109: note = Light chain variable domain 2
DIVLTQSPSSLSASVGDRVTITCRASQSISSYLNWYQQKPGKAPKLLIYAASSLQSGVPSRFSGSGSGTDFTLTISSLQPEDFATYYCQQSYSTPPYTFGQGTKLEIKR

SEQ ID NO: 63 - protein, 110 aa, synthetic construct
  REGION 1..110: note = Light chain variable domain 3
ELVLTQSPGTLSLSPGERATLSCRASQSIFNYVAWYQQKPGQAPRLLIYDASNRATGIPARFSGSGSGTDFTLTISSLEPEDFAVYYCQQRSKWPPTWTFGQGTRVDIKR

SEQ ID NO: 64 - protein, 109 aa, synthetic construct
  REGION 1..109: note = Light chain variable domain 4
ELVLTQSPGTLSLSPGERATLSCRASETVSSRQLAWYQQKPGQAPRLLIYGASSRATGIPDRFSGSGSGTDFTLTISRLEPEDSAVFYCQQYGSSPRTFGGGTKLEIKR

SEQ ID NO: 65 - protein, 109 aa, synthetic construct
  REGION 1..109: note = Light chain variable domain 5
ELVLTQSPGTLSLSPGERATLSCRASQSVSSSSLAWYQQKPGQAPRLLIYGASSRATGIPDRFSGSGSGTDFTLTISSLQPEDAATYYCQKYSSYPLTFGQGTKLEIKR

SEQ ID NO: 66 - protein, 109 aa, synthetic construct
  REGION 1..109: note = Light chain variable domain 6
ELVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 67 - protein, 109 aa, synthetic construct
  REGION 1..109: note = Light chain variable domain 7
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 68 - protein, 103 aa, synthetic construct
  REGION 1..103: note = Heavy chain constant domain
ASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 69 - protein, 107 aa, synthetic construct
  REGION 1..107: note = Light chain constant domain
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 70 - protein, 15 aa, synthetic construct
  REGION 1..15: note = Linker 2
GSAGSAPAPAGSGEF

SEQ ID NO: 71 - protein, 10 aa, synthetic construct
  REGION 1..10: note = Linker 3
GGGGSGGGGS

SEQ ID NO: 72 - protein, 5 aa, synthetic construct
  REGION 1..5: note = Linker 4
GGGGS

SEQ ID NO: 73 - protein, 5 aa, synthetic construct
  REGION 1..5: note = Linker 5
SGGGG

SEQ ID NO: 74 - protein, 5 aa, synthetic construct
  REGION 1..5: note = Linker 6
SRSSG

SEQ ID NO: 75 - protein, 5 aa, synthetic construct
  REGION 1..5: note = Linker 7
SGSSC

SEQ ID NO: 76 - protein, 12 aa, synthetic construct
  REGION 1..12: note = Linker 8
GKSSGSGSESKS

SEQ ID NO: 77 - protein, 6 aa, synthetic construct
  REGION 1..6: note = Linker 9
RPPPPC

SEQ ID NO: 78 - protein, 7 aa, synthetic construct
  REGION 1..7: note = Linker 10
SSPPPPC

SEQ ID NO: 79 - protein, 14 aa, synthetic construct
  REGION 1..14: note = Linker 11
GSTSGSGKSSEGKG

SEQ ID NO: 80 - protein, 18 aa, synthetic construct
  REGION 1..18: note = Linker 12
GSTSGSGKSSEGSGSTKG

SEQ ID NO: 81 - protein, 18 aa, synthetic construct
  REGION 1..18: note = Linker 13
GSTSGSGKPGSGEGSTKG

SEQ ID NO: 82 - protein, 14 aa, synthetic construct
  REGION 1..14: note = Linker 14
EGKSSGSGSESKEF

SEQ ID NO: 83 - protein, 15 aa, synthetic construct
  REGION 1..15: note = Linker 15
GSAGSAPAPAGSGEF

SEQ ID NO: 84 - protein, 10 aa, synthetic construct
  REGION 1..10: note = Linker 16
GGGGSGGGGS

SEQ ID NO: 85 - protein, 15 aa, synthetic construct
GGGGSGGGGSGGGGS

SEQ ID NO: 86 - DNA, 45 bp, synthetic construct
  source 1..45: mol_type = other DNA
ggcggtggaggtagtggaggaggcggtagtggtggcggtggtagt

SEQ ID NO: 87 - DNA, 45 bp, synthetic construct
  source 1..45: mol_type = other DNA
ggcggtggcggctcaggcggaggaggctccgggggaggtgggtcc

SEQ ID NO: 88 - protein, 7 aa, synthetic construct
DVPSGPG

SEQ ID NO: 89 - RNA, 21 nt, synthetic construct
  source 1..21: mol_type = other RNA
gatgtgcctagtggccctgga

SEQ ID NO: 90 - DNA, 21 bp, synthetic construct
  source 1..21: mol_type = other DNA
gacgttccatctggccctggt

SEQ ID NO: 91 - protein, 106 aa, synthetic construct
TVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 92 - DNA, 645 bp, synthetic construct
  source 1..645: mol_type = other DNA
gacatagttctcacccaaagtcccgggactctttcattgtcacccggagaaaccgcaacattgtcatgccgagcttcccaatcagtaggtagtaatttggcctggtatcaacaaaaaccaggccaggcaccccgccttctgatatatggagcctctaccggagccacaggtgtaccagcacgttttagcggttctcgttccgggacagactttactttgaccattacctcactgcagcctgaggacttcgctacatattattgccaacaatattattcttttctggccaaaacctttgggcaagggacccagttggagattaagcgaactgttgcagctcccagtgtttttatcttccctcccagtgatgaacaactgaaaagcggtaccgcctcagtggtttgtctccttaataatttctacccacgcgaagccaaggtgcaatggaaggttgataatgctttgcaatcagggaatagtcaggaatctgttacagaacaagattccaaggattcaacatatagcctgtctaataccctcactctgtccaaggctgattacgaaaagcacaaagtctatgcctgtgaggttactcaccaagggctgtcctcccctgttacaaaatctttcaatcggggagagagt

(19) United States
(12) Patent Application Publication
(10) Pub. No.: US 2026/0209300 A1
(43) Pub. Date: Jul. 23, 2026

(54) Fusion Protein Comprising Interleukin-2 Mutein and Antigen-Binding Fragment to Serum Albumin and Compositions and Uses Thereof

(71) Applicant: Aprilbio Co., Ltd., Chuncheon-si, Gangwon-do (KR)

(72) Inventor: Sang Hoon CHA, Chuncheon-si, Gangwon-do (KR)

(21) Appl. No.: 19/120,348

(22) PCT Filed: Oct. 19, 2023
(86) PCT No.: PCT/IB2023/060573

§ 371 Date: Apr. 11, 2025

(30) Foreign Application Priority Data
Oct. 19, 2022 (KR) 10-2022-0135135

(51) Int. Cl. C07K 14/55; A61K 38/00; A61P 37/06; C07K 16/18; C12N 15/70

(52) U.S. Cl. CPC C07K 14/55; A61P 37/06; C07K 16/18; C12N 15/70; A61K 38/00; C07K 2317/55; C07K 2317/565; C07K 2319/00

(57) ABSTRACT

Provided are recombinant fusion proteins and compositions and uses thereof, the recombinant fusion protein including an interleukin-2 mutein and an antigen-binding fragment to serum albumin. The recombinant fusion protein has the advantage of a relatively long administration cycle due to an increased in vivo half-life and can be effectively used for the treatment of various immune diseases and inflammatory neurological diseases including inflammatory bowel disease, systemic lupus erythematosus, etc., due to selective activity of the recombinant fusion protein on regulatory T cells.

### CROSS-REFERENCE TO RELATED APPLICATIONS

This application claims priority under 35 U.S.C. § 119 to Korean Appl. No. 10-2022-0135135, filed Oct. 19, 2022, the disclosure of which is incorporated by reference herein in its entirety.

### REFERENCE TO SEQUENCE LISTING SUBMITTED ELECTRONICALLY

This application contains a Sequence Listing which has been submitted in XML format via EFS-Web and is hereby incorporated by reference in its entirety. Said XML copy, created on Oct. 12, 2023, is named 2662-0008WO01_SEQL_ST26 and is 117,497 bytes in size.

### BACKGROUND

### 1. Field

The disclosure relates to fusion proteins comprising an interleukin-2 mutein and an antigen-binding fragment to serum albumin, and compositions and uses thereof.

### 2. Description of the Related Art

An autoimmune disease is a disease caused by autoimmunity when abnormalities occur in the immune system of the human body and refers to an erroneous response of the immune system to normal chemicals and some cells of the body. The human immune system basically recognizes microorganisms and cancer cells invading the human body as external antigens and has a strong ability to attack and remove them but does not attack its own cells due to self-tolerance. However, when the self-tolerance of the immune system is destroyed, the human body constantly destroys its own cells and causes inflammation and immune responses as autoreactive T cells that respond to its own cells (or self-antigens) are activated and autoantibodies are produced.

Although the causes of occurrence of autoimmune diseases have not been clearly identified, the causes are largely divided into genetic, acquired, and environmental factors. According to recent studies, autoimmune diseases cause a decrease in the number of regulatory T cells (Treg cells), and thus increasing Treg cells is considered an important therapeutic strategy.

Treg cells are a class of CD4+CD25+ T cells that suppress the activity of several immune cells. Treg cells are crucial for homeostasis of the immune system and play a major role in maintaining tolerance to self-antigens and regulating the immune response to foreign antigens. In many autoimmune and inflammatory diseases including systemic lupus erythematosus (SLE), inflammatory bowel disease (IBD), and graft-versus-host disease (GVHD), defects in the number or function of Treg cells were confirmed. Therefore, the development of therapies that activate the number and function of Treg cells is of great interest.

Interleukin 2 (IL-2) is known to play an important role in the activation of Treg cells, and Treg cells express IL-2 receptors, IL-2Rαβγ consisting of IL-2 receptor alpha subunit (IL-2RA, also called CD25), IL-2 receptor beta subunit (IL-2RB, also called CD122), and IL-2 receptor gamma subunit (IL-2RG, also called CD132), at a high constant level. ILTOO Company is carrying out clinical trials for autoimmune diseases such as type 1 diabetes and lupus by using low-dose IL-2, as a strategy to target only Treg cells. This is because Treg cells expressing IL-2Rαβγ respond to IL-2 at lower concentrations than many other immune cell types (Klatzmann D, 2015, Nat Rev Immunol. 15:283-94). However, there is a need for improvement in terms of safety, tolerability, and patient convenience even with such low-dose IL-2 administration, and thus the development of new mechanisms of non-toxic treatment is demanded.

IL-2 is a cytokine that is produced by CD4+ helper T cells and induces proliferation and activation of all T cells including Treg cells and NK cells. Among immune cells, Treg cells expressing IL-2 receptors (IL-2R) on the cell surface express an IL-2Rαβγ trimer, whereas CD4+ effector T cells, CD8+ effector T cells, and NK cells express an IL-2 receptor in the form of IL-2Rβγ.

Therefore, when developing an IL-2 mutein that selectively binds to the IL-2Rαβγ trimer rather than the IL-2Rβγ dimer, Treg cells can be selectively activated rather than CD8+ T cells and NK cells. As a result, the immune system of patients with abnormally overactive autoimmune diseases can be stabilized.

There is a need to develop fusion proteins for improved therapy of IL-2m and increased convenience to patients.

Antigen-binding fragments that bind to serum albumin are provided in U.S. Pat. Nos. 9,879,077 and 11,773,176, each incorporated herein by reference in its entirety.

### SUMMARY

Disclosed herein are recombinant fusion proteins comprising an interleukin-2 mutein (IL-2m) and an antigen binding fragment (Fab) that binds to serum albumin. In some embodiments, the IL-2m and the Fab to serum albumin are linked to each other by a linker. In some embodiments, a C-terminal region of a heavy chain of the Fab to serum albumin and the IL-2m are linked to each other by a linker. In other embodiments, the IL-2m, a linker, a heavy chain comprising a heavy chain region of the Fab to serum albumin, and a light chain comprising a light chain region of the Fab to serum albumin are linked by non-covalent bonds.

In some embodiments, the linker comprises 1 to 50 amino acids. In some embodiments, the linker comprises an amino acid sequence of any one of SEQ ID NOS: 16, 70-85, and 88. In some embodiments, the linker has an amino acid sequence of SEQ ID NO: 84.

The IL-2m, the linker, and the heavy chain can comprise an amino acid sequence of SEQ ID NO:3 or 4. The recombinant fusion protein can comprise the amino acid sequences of SEQ ID NO:3 or 4 and SEQ ID NO:13. The IL-2m can comprise an amino acid sequence having at least 90% identity to SEQ ID NO:1 or 2. The IL-2m can comprise an amino acid sequence of SEQ ID NO:1. The IL-2m can comprise an amino acid sequence of SEQ ID NO:2.

In some embodiments, the Fab can comprise
a heavy chain comprising a heavy chain variable domain comprising
   (a) a heavy chain complementarity determining domain 1 (CDR1) comprising the amino acid sequence of SYGIS (SEQ ID NO:22),
   a heavy chain complementarity determining domain 2 (CDR2) comprising the amino acid sequence of WINTYSGGTKYAQKFQG (SEQ ID NO:23), and
   a heavy chain complementarity determining domain 3 (CDR3) comprising the amino acid sequence of LGHCQRGICSDALDT (SEQ ID NO:24);
   (b) a heavy chain CDR1 comprising the amino acid sequence of SYGIS (SEQ ID NO:22),
   a heavy chain CDR2 comprising the amino acid sequence of RINTYNGNTGYAQRLQG (SEQ ID NO:25), and
   a heavy chain CDR3 comprising the amino acid sequence of LGHCQRGICSDALDT (SEQ ID NO:24);
   (c) a heavy chain CDR1 comprising the amino acid sequence of NYGIH (SEQ ID NO:26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
   (d) a heavy chain CDR1 comprising the amino acid sequence of SYAMS (SEQ ID NO:29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
   (e) a heavy chain CDR1 comprising the amino acid sequence of AYWIA (SEQ ID NO:32),
   a heavy chain CDR2 comprising the amino acid sequence of MIWPPDADARYSPSFQG (SEQ ID NO:33), and
   a heavy chain CDR3 comprising the amino acid sequence of LYSGSYSP (SEQ ID NO: 34); or
   (f) a heavy chain CDR1 comprising the amino acid sequence of AYSMN (SEQ ID NO:35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37); and
   a light chain comprising a light chain variable domain comprising
   (g) a light chain CDR1 comprising the amino acid sequence of RASQSISRYLN (SEQ ID NO:38),
   a light chain CDR2 comprising the amino acid sequence of GASRLES (SEQ ID NO: 39), and
   a light chain CDR3 comprising the amino acid sequence of QQSDSVPVT (SEQ ID NO:40);
   (h) a light chain CDR1 comprising the amino acid sequence of RASQSISSYLN (SEQ ID NO:41),
   a light chain CDR2 comprising the amino acid sequence of AASSLQS (SEQ ID NO: 42), and
   a light chain CDR3 comprising the amino acid sequence of QQSYSTPPYT (SEQ ID NO:43);
   (i) a light chain CDR1 comprising the amino acid sequence of RASQSIFNYVA (SEQ ID NO:44),
   a light chain CDR2 comprising the amino acid sequence of DASNRAT (SEQ ID NO: 45), and
   a light chain CDR3 comprising the amino acid sequence of QQRSKWPPTWT (SEQ ID NO:46);
   (j) a light chain CDR1 comprising the amino acid sequence of RASETVSSRQLA (SEQ ID NO:47),
   a light chain CDR2 comprising the amino acid sequence of GASSRAT (SEQ ID NO: 48), and
   a light chain CDR3 comprising the amino acid sequence of QQYGSSPRT (SEQ ID NO:49);
   (k) a light chain CDR1 comprising the amino acid sequence of RASQSVSSSSLA (SEQ ID NO:50),
   a light chain CDR2 comprising the amino acid sequence of GASSRAT (SEQ ID NO: 48), and
   a light chain CDR3 comprising the amino acid sequence of QKYSSYPLT (SEQ ID NO: 51); or
   (l) a light chain CDR1 comprising the amino acid sequence of RASQSVGSNLA (SEQ ID NO:52),
   a light chain CDR2 comprising the amino acid sequence of GASTGAT (SEQ ID NO: 53), and
   a light chain CDR3 comprising the amino acid sequence of QQYYSFLAKT (SEQ ID NO:54).

The Fab can comprises a heavy chain variable domain comprising a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and a light chain variable domain comprising a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. The heavy chain variable domain can comprise the amino acid sequence of SEQ ID NO:60. In some embodiments, the light chain variable domain comprises an amino acid sequence having at least 90% identity to SEQ ID NO:61, 62, 63, 64, 65, 66, or 67. The light chain variable domain can comprise the amino acid sequence of SEQ ID NO:67.

In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69 or 91. The Fab can comprise a heavy chain comprising the amino acid sequence of SEQ ID NO:6 and a light chain comprising the amino acid sequence of SEQ ID NO:91. The Fab can comprise a heavy chain constant 1 domain comprising the amino acid sequence of SEQ ID NO:68 linked to the heavy chain variable domain; and a light chain constant domain comprising the amino acid sequence of SEQ ID NO:69 or 91 linked to the light chain variable domain.

Disclosed herein are nucleic acids encoding the recombinant fusion protein disclosed herein. Also disclosed herein are expression vectors comprising the nucleic acid disclosed herein. Disclosed herein are cells transformed with the expression vectors disclosed herein.

Disclosed herein are compositions comprising the recombinant fusion proteins disclosed herein and a carrier. Also disclosed herein are pharmaceutical compositions comprising the recombinant fusion proteins disclosed herein and pharmaceutically acceptable carriers. Disclosed herein are kits comprising the compositions or pharmaceutical compositions disclosed herein and labels comprising instructions for uses.

Disclosed herein are methods of treating for preventing or treating autoimmune disease or inflammatory neurological disease in a subject in need thereof, comprising administering the pharmaceutical compositions disclosed herein to the subject. Also disclosed herein are uses of a pharmaceutical compositions for preventing or treating autoimmune disease or inflammatory neurological disease, comprising the recombinant fusion protein disclosed herein as an active ingredient. In some embodiments, the autoimmune disease is selected from the group consisting of Crohn's disease, ulcerative colitis, spondylitis ankylopoietica, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, restenosis of a blood vessel, glomerulonephritis, and gastrointestinal allergy, and the inflammatory neurological disease is selected from the group consisting of amyotrophic lateral sclerosis and Parkinson's disease.

Additional aspects will be set forth in part in the description which follows and, in part, will be apparent from the description, or can be learned by practice of the presented embodiments of the disclosure.

According to some aspects of the disclosure, a recombinant fusion protein includes an interleukin-2 mutein and an antigen-binding fragment to serum albumin. In some embodiments, the recombinant fusion protein consists of a heavy chain including 366 amino acids and a light chain having 215 amino acids. In addition, the recombinant fusion protein has no glycosylation in the antigen-binding fragment to serum, and whereas IL-2 protein has one N—O-glycosylation site. Therefore, the recombinant fusion protein according to some aspects can include glycosylation.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A shows a heavy chain expression vector and FIG. 1B shows a light chain expression vector, for preparing a recombinant fusion protein;

FIG. 2 shows a structure of a SAFA-IL-2m (Fab-IL-2m);

FIGS. 3A and 3B show the size of SAFA-IL-2m and efavaleukin-alfa analogue proteins according to an aspect in 1 μg/well under reducing (R), non-reducing (NR(B)) with heat applied, and non-reducing (NR(NB)) without heat applied conditions by SDS-PAGE;

FIGS. 4A and 4B show the purity of SAFA-IL-2m2 and efavaleukin-alfa analogue proteins by SE-HPLC;

FIGS. 5A-5C show the binding ability of SAFA-IL-2m to human serum albumin, monkey serum albumin, and mouse serum albumin;

FIGS. 6A and 6B are graphs of measurement of activity by SAFA-IL2m in a HEK-blue IL-2 reporter cell line;

FIGS. 7A and 7B show the binding activity of SAFA-IL-2m to CD25 and CD122;

FIG. 8 shows graphs of phospho-STAT5 signaling measurement of a SAFA-IL-2m dose-response in regulatory T cells, CD4+ Tconv cells, CD8+ T cells, and NK cells isolated from mouse spleens;

FIG. 9 shows graphs of phospho-STAT5 signaling measurement of a SAFA-IL-2m dose-response in regulatory T cells, CD4+ Tconv cells, CD8+ T cells, and NK cells using human peripheral blood mononuclear cells;

FIG. 10 shows graphs of Ki67 proliferation measurement of a dose-response of SAFA-IL-2m in regulatory T cells, CD4+ Tconv cells, CD8+ T cells, and NK cells using human peripheral blood mononuclear cells;

FIG. 11 shows graphs of the ratio of the number of regulatory T cells to the number of CD4+ Tconv cells, the ratio of the number of regulatory T cells to the number of CD8+ T cells, and the ratio of the number of regulatory T cells to the number of NK cells, after administering a single intraperitoneal injection and isolating cells from the spleen, inguinal lymph node, and mesenteric lymph node of mice treated with an SAFA-IL-2m and a positive control group;

FIG. 12 is a graph of the immune response of SAFA-IL-2m in immunized marmoset monkeys;

FIGS. 13A and 13B are graphs of the disease activity index (DAI) scores, changes in the intestinal length, and cytokine levels in mouse models having dextran sulfate sodium (DSS)-induced inflammatory bowel disease, after the administration of a SAFA-IL-2m and a positive control to the mouse models;

FIGS. 14A and 14B show changes in the blood concentration of SAFA-IL-2m over time, after intravenous and subcutaneous administration of SAFA-IL-2m to monkeys;

FIG. 15 shows changes in the blood concentration of SAFA-IL-2m over time, after intravenous administration of SAFA-IL-2m to mice;

FIGS. 16A and 16B show changes in the proliferation of regulatory T cells over time, after subcutaneous administration of SAFA-IL-2m to monkeys;

FIGS. 17A and 17B show the bio-distribution of SAFA-IL-2m, after subcutaneously administering SAFA-IL-2m to a collagen-induced arthritis mouse model;

FIGS. 18A-18C show the level of proteinuria scores, lymphadenopathy scores, and levels of anti-dsDNA IgG in blood, after administering SAFA-IL-2m to an MRL/lpr mouse model of systemic lupus erythematosus; and

FIGS. 19A and 19B show the hematoxylin & eosin (H&E) staining and immunofluorescence staining for IgG targeting the kidney tissue obtained from an MRL/lpr mouse model of systemic lupus erythematosus, after administering SAFA-IL-2m according to an aspect to the mouse model.

### DETAILED DESCRIPTION

The term “interleukin-2 mutein (IL-2m)” as used in the present specification refers to a mutation of IL-2, which selectively binds to an IL-2 receptor trimer, IL-2Rαβγ, expressed in regulatory T cells rather than an IL receptor dimer, IL-2Rβγ expressed in CD8⁺ effector T cells or NK cells. In some embodiments, the IL-2m can include an amino acid sequence of SEQ ID NO: 2.

The IL-2m, the linker, and the heavy chain can comprise an amino acid sequence of SEQ ID NO:3 or 4. In some embodiments, the IL-2m, the linker, and the heavy chain can comprise an amino acid sequence encoded by a nucleotide sequence of SEQ ID NO:5. The recombinant fusion protein can comprise the amino acid sequences of SEQ ID NO:3 or 4 and SEQ ID NO:2. The IL-2m can comprise an amino acid sequence having at least 90% identity to SEQ ID NO:1 or 2. The IL-2m can comprise an amino acid sequence of SEQ ID NO:1. The IL-2m can comprise an amino acid sequence of SEQ ID NO:2.

The term “serum albumin” as used in the present specification is one of proteins constituting the basic substance of cells and plays an important role in maintaining the osmotic pressure between blood vessels and tissues by allowing body fluids to stay in blood vessels. In addition, the term “antigen-binding fragment to serum albumin” as used in the present specification refers to an anti-serum albumin antibody or an antigen-binding fragment of an antibody molecule, each binding specifically to an epitope of serum albumin. An antigen-binding fragment of an antibody or an antibody fragment refers to a fragment having an antigen-binding function, and includes Fab, F(ab′), F(ab′)2, Fv, and the like. Among the antibody fragments, Fab has a structure having variable regions of light and heavy chains, constant regions of a light chain, and first constant regions of a heavy chain (CH1), and that is, has one antigen-binding site. Fab′ differs from Fab in that it has a hinge region including one or more cysteine residues at the C-terminus of the CH1 domain of the heavy chain. F(ab′)2 antibody is produced by forming a disulfide bond between cysteine residues in the hinge region of Fab′. Fv is a minimal antibody fragment having only a variable region of the heavy chain and a variable region of the light chain, and recombinant technology for generating Fv fragments is disclosed in PCT International Patent Publications WO88/10649, WO88/106630, WO88/07085, WO88/07086, and WO88/09344. A two-chain Fv includes a variable region of the heavy chain and a variable region of the light chain that are linked to each other by a non-covalent bond, and a single-chain Fv (scFv) generally includes a variable region of the heavy chain and a variable region of the light chain that are covalently linked via a peptide linker or directly linked at the C-terminus to form a dimer-like structure as in the double-chain Fv. Such antibody fragments can be obtained by using proteolytic enzymes (for example, Fab can be obtained by restriction digestion of whole antibodies with papain, and F(ab′)2 fragment can be obtained by cleavage with pepsin) or can be also prepared by recombinant gene technology.

In some embodiments, the antigen-binding fragment to serum albumin can include: a light chain region including an amino acid sequence of SEQ ID NO:13; and a heavy chain region including an amino acid sequence of SEQ ID NO:1.

In some embodiments, the Fab heavy chain can comprise a leader amino acid sequence of SEQ ID NO:19, which is cleaved after the production of the fusion protein, i.e., the final fusion protein does not contain SEQ ID NO:19. The Fab heavy chain can comprise a leader amino acid sequence encoded by the nucleotide sequence of SEQ ID NO:20.

In some embodiments, the Fab light chain can comprise a leader amino acid sequence of SEQ ID NO:21, which is cleaved after the production of the fusion protein, i.e., the final fusion protein does not contain SEQ ID NO:21.

The term “recombinant fusion protein” as used in the present specification refers to a protein in which two or more types of proteins are artificially linked. In some embodiments, the term refers to a protein in which the IL-2 protein is linked to the antigen-binding fragment to serum albumin, i.e., anti-serum albumin Fab antibody fragment. Such a recombinant fusion protein can be obtained by chemical synthesis or expression and purification by genetic recombination methods, after each partner is determined. In some embodiments, the recombinant fusion protein can be obtained by expressing a fusion gene (expression vector), in which sequences of a gene encoding the IL-2 protein are linked to sequences of a gene encoding the anti-serum albumin Fab antibody fragment, in a cell expression system. In such a recombinant fusion protein, the IL-2 protein and the anti-serum albumin Fab antibody fragment can be linked directly to each other or linked to each other through a connector such as a linker. In some embodiments, the recombinant fusion protein can be the one in which the IL-2 protein, a linker, a heavy chain including a heavy chain region of the antigen-binding fragment to serum albumin, and a light chain including a light chain region of the antigen-binding fragment to serum albumin are linked through non-covalent bonds. For example, the recombinant fusion protein can include a peptide having an amino acid sequence of SEQ ID NO:3 or 4 and a peptide having an amino acid sequence of SEQ ID NO:13.

The term “linker” as used in the present specification refers to a peptide inserted between proteins to enhance the activity of each protein, wherein the each protein is linked by increasing the structural flexibility thereof in the preparation of the recombinant fusion portion by linking the IL-2 protein and the anti-serum albumin Fab antibody fragment. As long as the linker can minimize an immune response, the type of linker or number of amino acids is not limited. For example, the linker can include 1 to 20 amino acids, 1 to 15 amino acids, 1 to 10 amino acids, or 1 to 8 amino acids. In some embodiments, the linker can link the IL-2 protein to the C-terminus of the heavy chain region of the antigen-binding fragment to serum albumin. For example, the linker can include an amino acid of SEQ ID NO:84.

Disclosed herein are recombinant fusion proteins comprising an interleukin-2 mutein (IL-2m) and an antigen binding fragment (Fab) that binds to serum albumin. In some embodiments, the IL-2m and the Fab to serum albumin are linked to each other by a linker. In some embodiments, a C-terminal region of a heavy chain of the Fab to serum albumin and the IL-2m are linked to each other by a linker. In other embodiments, the IL-2m, a linker, a heavy chain comprising a heavy chain region of the Fab to serum albumin, and a light chain comprising a light chain region of the Fab to serum albumin are linked by non-covalent bonds.

In some embodiments, the linker comprises 1 to 50 amino acids. In some embodiments, the linker comprises an amino acid sequence of any one of SEQ ID NOS: 16, 70-85, and 88. In some embodiments, the linker has an amino acid sequence of SEQ ID NO: 84.

The IL-2m, the linker, and the heavy chain can comprise an amino acid sequence of SEQ ID NO:3 or 4. The recombinant fusion protein can comprise the amino acid sequences of SEQ ID NO:3 or 4 and SEQ ID NO:13. The IL-2m can comprise an amino acid sequence having at least 90% identity to SEQ ID NO:2. The IL-2m can comprise an amino acid sequence of SEQ ID NO:2. The IL-2m can comprise an amino acid sequence of SEQ ID NO:1.

In some embodiments, the Fab can comprise
a heavy chain comprising a heavy chain variable domain comprising
   a heavy chain complementarity determining domain 1 (CDR1) comprising the amino acid sequence of SYGIS (SEQ ID NO:22), a heavy chain complementarity determining domain 2 (CDR2) comprising the amino acid sequence of WINTYSGGTKYAQKFQG (SEQ ID NO:23), and a heavy chain complementarity determining domain 3 (CDR3) comprising the amino acid sequence of LGHCQRGICSDALDT (SEQ ID NO:24);
   a heavy chain CDR1 comprising the amino acid sequence of SYGIS (SEQ ID NO:22),
   a heavy chain CDR2 comprising the amino acid sequence of RINTYNGNTGYAQRLQG (SEQ ID NO:25), and
   a heavy chain CDR3 comprising the amino acid sequence of LGHCQRGICSDALDT (SEQ ID NO:24);
   a heavy chain CDR1 comprising the amino acid sequence of NYGIH (SEQ ID NO:26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
   a heavy chain CDR1 comprising the amino acid sequence of SYAMS (SEQ ID NO:29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
   a heavy chain CDR1 comprising the amino acid sequence of AYWIA (SEQ ID NO:32),
   a heavy chain CDR2 comprising the amino acid sequence of MIWPPDADARYSPSFQG (SEQ ID NO:33), and
   a heavy chain CDR3 comprising the amino acid sequence of LYSGSYSP (SEQ ID NO: 34); or
   a heavy chain CDR1 comprising the amino acid sequence of AYSMN (SEQ ID NO:35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37); and
   a light chain comprising a light chain variable domain comprising
   a light chain CDR1 comprising the amino acid sequence of RASQSISRYLN (SEQ ID NO:38),
   a light chain CDR2 comprising the amino acid sequence of GASRLES (SEQ ID NO: 39), and
   a light chain CDR3 comprising the amino acid sequence of QQSDSVPVT (SEQ ID NO:40);
   a light chain CDR1 comprising the amino acid sequence of RASQSISSYLN (SEQ ID NO:41),
   a light chain CDR2 comprising the amino acid sequence of AASSLQS (SEQ ID NO: 42), and
   a light chain CDR3 comprising the amino acid sequence of QQSYSTPPYT (SEQ ID NO:43);
   a light chain CDR1 comprising the amino acid sequence of RASQSIFNYVA (SEQ ID NO:44),
   a light chain CDR2 comprising the amino acid sequence of DASNRAT (SEQ ID NO: 45), and
   a light chain CDR3 comprising the amino acid sequence of QQRSKWPPTWT (SEQ ID NO:46);
   a light chain CDR1 comprising the amino acid sequence of RASETVSSRQLA (SEQ ID NO:47),
   a light chain CDR2 comprising the amino acid sequence of GASSRAT (SEQ ID NO: 48), and
   a light chain CDR3 comprising the amino acid sequence of QQYGSSPRT (SEQ ID NO:49);
   a light chain CDR1 comprising the amino acid sequence of RASQSVSSSSLA (SEQ ID NO:50),
   a light chain CDR2 comprising the amino acid sequence of GASSRAT (SEQ ID NO: 48), and
   a light chain CDR3 comprising the amino acid sequence of QKYSSYPLT (SEQ ID NO: 51); or
   a light chain CDR1 comprising the amino acid sequence of RASQSVGSNLA (SEQ ID NO:52),
   a light chain CDR2 comprising the amino acid sequence of GASTGAT (SEQ ID NO: 53), and
   a light chain CDR3 comprising the amino acid sequence of QQYYSFLAKT (SEQ ID NO:54).

In some embodiments, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the light chain variable domain comprises an amino acid sequence having at least 90% identity to SEQ ID NO:61, 62, 63, 64, 65, 66, or 67. In some embodiments, the heavy chain variable domain comprises an amino acid sequence of SEQ ID NO:55, 56, 57, 58, 59, or 60, and the light chain variable domain comprises an amino acid sequence of SEQ ID NO: 61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69 or 91.

In some embodiments, the heavy chain of the Fab comprises an amino acid sequence of SEQ ID NO:6. In some embodiments, the fusion protein comprises an amino acid sequence of SEQ ID NO:13 and an amino acid sequence of SEQ ID NO:6.

In some embodiments of the recombinant proteins disclosed herein, the heavy chain variable domain comprises a heavy chain CDR1 comprising the amino acid sequence of SEQ ID NO: 35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90%, at least 93%, at least 95%, at least 96%, at least 97%, at least 98%, at least 99%, or 100% identity to SEQ ID NO:55, 56, 57, 58, 59, or 60.

In some embodiments, the light chain variable domain comprises an amino acid sequence having at least 90%, at least 93%, at least 95%, at least 96%, at least 97%, at least 98%, at least 99%, or 100% identity to SEQ ID NO:61, 62, 63, 64, 65, 66, or 67.

In some embodiments, the heavy chain variable domain comprises the amino acid sequence of SEQ ID NO:55, 56, 57, 58, 59, or 60, and the light chain variable domain comprises the amino acid sequence of SEQ ID NO:61, 62, 63, 64, 65, 66 or 67.

In some embodiments, the Fab comprises a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60, and a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, or 66 or 67, respectively, or in any combinations of heavy chain variable domain and light chain variable domain disclosed herein. For example, the Fab can comprise a heavy chain variable domain comprising an amino acid sequence having at least 90%, at least 93%, at least 95%, at least 96%, at least 97%, at least 98%, at least 99%, or 100% identity to SEQ ID NO:60 and a light chain variable domain comprising an amino acid sequence having at least 90%, at least 93%, at least 95%, at least 96%, at least 97%, at least 98%, at least 99%, or 100% identity to SEQ ID NO:67.

In some embodiments, the heavy chain constant domain comprises an amino acid sequence having at least 90%, at least 93%, at least 95%, at least 96%, at least 97%, at least 98%, at least 99%, or 100% identity to SEQ ID NO:68.

In some embodiments, the light chain constant domain comprises an amino acid sequence having at least 90%, at least 93%, at least 95%, at least 96%, at least 97%, at least 98%, at least 99%, or 100% identity to SEQ ID NO:69 or 91.

In some embodiments, the recombinant fusion protein can comprise a heavy chain comprising an amino acid sequence having at least 90%, at least 93%, at least 95%, at least 96%, at least 97%, at least 98%, at least 99%, or 100% identity to SEQ ID NO:3 or 4. In some embodiments, the Fab comprises a heavy chain domain comprising an amino acid sequence having at least 90%, at least 93%, at least 95%, at least 96%, at least 97%, at least 98%, at least 99%, or 100% identity to SEQ ID NO:6 (V_{H}-C_{HI} domain). In some embodiments, the Fab comprises a light chain domain comprising an amino acid sequence having at least 90%, at least 93%, at least 95%, at least 96%, at least 97%, at least 98%, at least 99%, or 100% identity to SEQ ID NO:13 (V_{L}-C_{κ} domain).

In some embodiments, the recombinant fusion protein can comprise a heavy chain comprising an amino acid sequence having at least 90%, at least 93%, at least 95%, at least 96%, at least 97%, at least 98%, at least 99%, or 100% identity to SEQ ID NO:3 or 4; and a light chain comprising an amino acid sequence having at least 90%, at least 93%, at least 95%, at least 96%, at least 97%, at least 98%, at least 99%, or 100% identity to SEQ ID NO:13. The recombinant protein can have significantly improved pharmacokinetic properties while maintaining the intrinsic biological activity of the IL-2m.

In some embodiments, the Fab comprises a heavy chain domain comprising an amino acid sequence having at least 90%, at least 93%, at least 95%, at least 96%, at least 97%, at least 98%, at least 99%, or 100% identity to SEQ ID NO:6 (V_{H}-C_{HI} domain) and a light chain domain comprising an amino acid sequence having at least 90%, at least 93%, at least 95%, at least 96%, at least 97%, at least 98%, at least 99%, or 100% identity to SEQ ID NO: 13 (V_{L}-C_{κ} domain).

In some embodiments, the variable domain of the heavy chain (VH) can include a heavy chain complementarity-determining region (HCDR) comprising of amino acid sequences of SEQ ID NOS: 35, 36, 37. For example, the variable domain of the heavy chain (VH) can include an amino acid sequence of SEQ ID NO:60 or an amino acid sequence having at least 80%, 85%, 90%, 95%, 99% or more sequence identity with the amino acid sequence of SEQ ID NO:60. The C-terminus of the variable domain of the heavy chain (VH) can be linked to the constant 1 domain of the heavy chain (CH1). Here, the constant 1 domain of the heavy chain (CH1) can include an amino acid sequence of SEQ ID NO:68, and for example, can include a sequence having at least 80%, 85%, 90%, 95%, 99% or more sequence identity with the amino acid sequence of SEQ ID NO:68, but embodiments are not particularly limited thereto.

In some embodiments, the variable domain of the light chain (VL) can include a light chain complementarity-determining region (LCDR) comprising of amino acid sequences of SEQ ID NOS: 52, 53, and 54. For example, the variable domain of the light chain (VL) can include an amino acid sequence of SEQ ID NO:67 or an amino acid sequence having at least 80%, 85%, 90%, 95%, 99% or more sequence identity with the amino acid sequence of SEQ ID NO: 67. The C-terminus of the variable domain of the light chain (VL) can be linked to the constant domain of the light chain (CL), wherein the light chain constant domain (CL) can include an amino acid sequence of SEQ ID NO:69 or 91, and for example, can include a sequence having at least 80%, 85%, 90%, and 95% or more sequence identity with the amino acid sequence of SEQ ID NO:69 or 91, but embodiments are not particularly limited thereto.

The term “identity” as used in the present specification refers to the overall relevance between polymer molecules, such as nucleic acids (e.g., DNA molecules and/or RNA molecules), and/or between polypeptides. For example, polypeptides are considered “substantially identical” when amino acid sequences thereof have at least 60%, 65%, 70%, 75%, 80%, 85%, 90%, 95%, or 99% identity. Calculating the percent identity of two nucleic acid or polypeptide sequences can be performed by, for example, aligning two sequences for the optimal comparison purposes (e.g., a gap can be introduced in one or both of first and second sequences for the optimal alignment, and non-identical sequences can be ignored for the comparison purposes). For example, the length of a sequence aligned for the comparison purposes is at least 60%, at least 70%, at least 80%, at least 90%, at least 95%, or substantially 100% of the length of a reference sequence. Subsequently, the nucleic acid or polypeptide sequences at corresponding positions are then compared. Determination of the percent identity between the two sequences and comparison of these sequences can be accomplished by using mathematical algorithms. As is well known to those skilled in the art, the amino acid or nucleic acid sequences can be compared by using any of a variety of algorithms including those available in commercial computer programs, specifically BLASTN for nucleotide sequences, and BLASTP, gapped BLAST, and PSIBLAS for amino acid sequences.

In some embodiments, in the fusion proteins disclosed herein, the heavy chain and the light chain of the Fab are bound by a noncovalent bond.

In some embodiments, the antigen-binding fragment that binds to serum albumin can be linked to the IL-2m through a linker. For example, the linker can link the IL02m to any one region selected from the C-terminus of the constant 1 domain of the heavy chain (CH1), the N-terminus of the variable domain of the heavy chain (VH), the C-terminus of the constant domain of the light chain (CL), and the N-terminus of the variable domain of the light chain (VL). In addition, the linker can be changed for use as necessary.

For example, the linker can be a polypeptide comprising 5 to 400 amino acids, 5 to 200 amino acids, or 5 to 200 amino acids. Such a peptide linker can include Gly, Asn and Ser residues, and can also include neutral amino acids such as Thr and Ala. The amino acid sequences suitable for the peptide linker are known in the art. In addition, the number of copies “n” can be adjusted in consideration of linker optimization to achieve proper separation between functional moieties or to maintain essential inter-moiety interactions. Other linkers are known in the art, and for example, a G-S linker, in which not only a polar amino acid residue is added to improve water solubility, but also amino acid residues, such as T and A, are added to maintain flexibility, can be used.

Therefore, the linker can be a flexible linker containing G, S, and/or T, A residues. The linker can have a general formula of (GpSs)ₙ or (SpGs)ₙ, wherein, independently, p is an integer of 1 to 10, s is 0 or an integer of 0 to 10, p+s is an integer of 20 or less, and n is an integer of 1 to 20. More specifically, examples of the linker can include (GGGGS)ₙ (SEQ ID NO:72), (SGGGG)ₙ (SEQ ID NO:73), (SRSSG)ₙ (SEQ ID NO:74), (SGSSC)ₙ (SEQ ID NO:75), (GKSSGSGSESKS)ₙ (SEQ ID NO:76), (RPPPPC)ₙ (SEQ ID NO:77), (SSPPPPC)ₙ (SEQ ID NO: 78), (GSTSGSGKSSEGKG)ₙ (SEQ ID NO:79), (GSTSGSGKSSEGSGSTKG)ₙ (SEQ ID NO: 80), (GSTSGSGKPGSGEGSTKG)ₙ (SEQ ID NO:81), or (EGKSSGSGSESKEF)ₙ (SEQ ID NO: 82), wherein n can be an integer of 1 to 20, or 1 to 10.

In addition, the linker can comprise an amino acid sequence of SEQ ID NO:84, 85 or 88, but embodiments are not particularly limited thereto.

In some embodiments, the linker comprises 1 to 50 amino acids. In some embodiments, the linker comprises an amino acid sequence of any one of SEQ ID NOS: 16, 70-85, and 88. In some embodiments, the recombinant fusion protein can include: i) a heavy chain including a heavy chain fragment SL335 of SEQ ID NO:6, a linker of SEQ ID NO:84, and an IL-2m of SEQ ID NO:1; and a light chain including a light chain fragment SL335 of SEQ ID NO:69 or 91, or ii) a heavy chain including a heavy chain fragment SL335 of SEQ ID NO: 6, a linker of SEQ ID NO:84, and IL-2m of SEQ ID NO:2; and a light chain including a light chain fragment SL335 of SEQ ID NO:69 or 91. The recombinant fusion protein can have significantly improved pharmacokinetic properties while maintaining the inherent biological activity of the IL-2m.

In some embodiments, recombinant fusion proteins (SAFA-IL-2m) are prepared, including: the antigen-binding fragment that binds to serum albumin; and the hIL-2m each fused to the C-terminus of each of the constant 1 domain of the heavy chain (CH1) and the constant domain of the light chain (CL). As the recombinant fusion protein retains the biological activity of each factor, specifically, binding ability to each of human serum albumin and IL-2m receptors, the half-life of in vivo loss can be extended, and the therapeutic efficacy such as growth promotion of the ovary can be improved.

In some embodiments, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and wherein the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence of SEQ ID NO:55, 56, 57, 58, 59, or 60, and the light chain variable domain comprises an amino acid of SEQ ID NO:61, 62, 63, 64, 65, 66, or 67.

In some embodiments, the light chain constant domain comprises an amino acid sequence having at least 90% identity to SEQ ID NO:69 or 91.

In some embodiments, the Fab comprises a heavy chain comprising the amino acid sequence of SEQ ID NO:6 and a light chain comprising the amino acid sequence of SEQ ID NO: 13. In some embodiments, the Fab comprises a heavy chain constant 1 domain comprising the amino acid sequence of SEQ ID NO:68 linked to the heavy chain variable domain; and a light chain constant domain comprising the amino acid sequence of SEQ ID NO: 69 or 91 linked to the light chain variable domain.

An antigen binding fragment of an antibody or an antibody fragment refers to a fragment retaining an antigen-binding function, and includes Fab, F(ab′), F(ab′)2, Fv, etc. Fab of the antibody fragments has a structure including variable regions of a light chain and a heavy chain, a constant region of the light chain, and a constant region (CH) of the heavy chain with one antigen-binding site. Fab′ differs from Fab in that it has a hinge region containing one or more cysteine residues at the C-terminal of the heavy chain CH domain. F(ab′)2 antibody is produced when the cysteine residue of the hinge region of Fab′ forms a disulfide bond. Recombinant techniques for generating Fv fragments with minimal antibody fragments having only a heavy chain variable region and a light chain variable region are described in PCT International Publication Nos. WO88/10649, WO88/106630, WO88/07085, WO88/07086, and WO88/09344. In a two-chain Fv, a heavy chain variable region and a light chain variable region are connected via a non-covalent bond. In a single chain Fv (scFv), a heavy chain variable region and a light chain variable region are generally connected via a peptide linker by a covalent bond or directly at the C-terminal. Thus, the single chain Fv (scFv) can have a structure such as a dimer, like the two-chain Fv. Such an antibody fragment can be obtained using a protein hydrolyzing enzyme (for example, when a whole antibody is cleaved with papain, Fab can be obtained, and when a whole antibody is cleaved with pepsin, F(ab′)2 fragment can be obtained), and it can also be produced through a recombinant gene technology.

In some embodiments, the antigen binding fragment against serum albumin can include a heavy chain region comprising an amino acid sequence of SEQ ID NO:6; and a light chain region comprising an amino acid sequence of SEQ ID NO:13. In some embodiments, nucleic acid molecule encoding the heavy chain region comprising the amino acid sequence of SEQ ID NO: 6 can have a nucleotide sequence of SEQ ID NO:7 or 8. In some embodiments, nucleic acid molecule encoding the light chain region comprising the amino acid sequence of SEQ ID NO: 13 can have a nucleotide sequence of SEQ ID NO:14 or 15.

The Fab can comprises a heavy chain variable domain comprising a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and a light chain variable domain comprising a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. The heavy chain variable domain can comprise the amino acid sequence of SEQ ID NO:60. In some embodiments, the light chain variable domain comprises an amino acid sequence having at least 90% identity to SEQ ID NO:61, 62, 63, 64, 65, 66, or 67. The light chain variable domain can comprise the amino acid sequence of SEQ ID NO:67.

In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69 or 91. The Fab can comprise a heavy chain comprising the amino acid sequence of SEQ ID NO:6 and a light chain comprising the amino acid sequence of SEQ ID NO:91. The Fab can comprise a heavy chain constant 1 domain comprising the amino acid sequence of SEQ ID NO:68 linked to the heavy chain variable domain; and a light chain constant domain comprising the amino acid sequence of SEQ ID NO:69 or 91 linked to the light chain variable domain.

As used herein, the terms “antibody” and “antibodies” are terms of art and can be used interchangeably herein and refer to a molecule with an antigen-binding site that specifically binds an antigen. Antibodies can include, e.g., monoclonal antibodies, recombinantly produced antibodies, human antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multispecific antibodies.

Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule.

As used herein, the terms “bioeffector moiety,” “antigen-binding domain,” “antigen-binding region,” “antigen-binding site,” and similar terms refer to the portions of the recombinant protein that comprises the amino acid residues that confer on the recombinant protein its specificity for the antigen (e.g., the complementarity determining regions (CDR)). The antigen-binding region can be derived from any animal species, such as feline, rodents (e.g., mouse, rat, or hamster) and humans.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody. The terms “VH” and “VH domain” are used interchangeably to refer to the heavy chain variable region of an antibody.

As used herein, the term “heavy chain (HC or CH)” refers to both a full-length heavy chain and a fragment thereof, the full-length heavy chain including a variable region domain VH including an amino acid sequence having a sufficient variable region (VR) sequence to confer specificity for an antigen and three constant region domains CH1, CH2, and CH3. As used herein, the term “light chain (LC or CL)” refers to both a full-length light chain and a fragment thereof, the full-length light chain including a variable region domain VL including an amino acid sequence having a sufficient VR sequence to confer specificity for an antigen and a constant region domain CL.

The heavy chain constant domain and the light chain constant domain can be derived from an IgG1 antibody constant domain, and in any one or more thereof, cysteine which is an amino acid used in a disulfide bond between the light chain and the heavy chain domain can be conserved or deleted or substituted with an amino acid residue other than cysteine. For example, the heavy chain constant domain can comprise an amino acid sequence having at least 90%, at least 93%, at least 95%, at least 96%, at least 97%, at least 98%, at least 99%, or 100% identity to SEQ ID NO:68, and the light chain constant domain can comprise an amino acid sequence having at least 90%, at least 93%, at least 95%, at least 96%, at least 97%, at least 98%, at least 99%, or 100% identity to SEQ ID NO:69 or 91. The deletion or substitution of cysteine in the domain can contribute to improving an expression level of the recombinant protein in transformed cells during a process of producing the above-mentioned recombinant protein. In some embodiments, (i) one or more cysteines in the heavy chain constant domain and/or (ii) one or more cysteines in the light chain constant domain that is/are located in an interchain disulfide bond between the light chain and the heavy chain is/are conserved, deleted, and/or substituted with an amino acid residue other than cysteine.

As used herein, the term “constant region” or “constant domain” are interchangeable and have its meaning common in the art. The constant region is an antibody portion, e.g., a carboxyl terminal portion of a light and/or heavy chain, which is not directly involved in binding of an antibody to an antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (Cκ) or lambda (Cλ) based on the amino acid sequence of the constant domains. Light chain amino acid sequences are well known in the art. In specific embodiments, the light chain is a human light chain.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore® or KinExA.

In some embodiments, the binding affinity of the recombinant fusion proteins disclosed herein has a binding affinity that is at least 1.5-fold, at least 2-fold, at least 3-fold, at least 4-fold, at least 5-fold, at least 6-fold, at least 7-fold, at least 8-fold, at least 9-fold, at least 10-fold, or any ranges therein higher than that of human IL-2m, e.g., 2-fold to 10-fold higher.

As used herein, an “epitope” is a term in the art and refers to a localized region of an antigen to which an antibody can specifically bind. An epitope can be, e.g., contiguous amino acids of a polypeptide (linear or contiguous epitope) or an epitope can, e.g., come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligopeptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson, A. (1976) J. Biol. Chem. 251:6300-6303). Antibody: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques. In some embodiments, the epitope of an antibody is determined using alanine scanning mutagenesis studies.

As used herein, the terms “immunospecifically binds,” “immunospecifically recognizes,” “specifically binds,” and “specifically recognizes” are analogous terms in the context of antibodies and refer to molecules that bind to an antigen (e.g., epitope, immune complex, or binding partner of an antigen-binding site) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In some embodiments, molecules that immunospecifically bind to an antigen bind to the antigen with a K_{A} that is at least 2 logs, 2.5 logs, 3 logs, 4 logs or greater than the K_{A} when the molecules bind to another antigen.

In some embodiments, molecules that immunospecifically bind to an antigen do not cross react with other proteins under similar binding conditions. In some embodiments, molecules that immunospecifically bind to an antigen do not cross react with other proteins. In some embodiments, provided herein are recombinant proteins that bind to a specified antigen with higher affinity than to another unrelated antigen. In certain embodiments, provided herein is a recombinant protein that binds to a specified antigen (e.g., human serum albumin) with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In some embodiments, the extent of binding of a recombinant protein described herein to an unrelated, protein is less than 10%, 15%, or 20% of the binding of the antibody to the specified antigen as measured by, e.g., a radioimmunoassay.

In some embodiments, provided herein are recombinant proteins that bind to an antigen of various species, such as feline, rodents (e.g., mouse, rat, or hamster) and humans. In some embodiments, provided herein are recombinant proteins that bind to a human antigen with higher affinity than to another species of the antigen. In certain embodiments, provided herein are recombinant proteins that bind to a human antigen with a 5%, 10%, 15%, 20%, 25%, 30%, 35%, 40%, 45%, 50%, 55%, 60%, 65%, 70% or higher affinity than to another species as measured by, e.g., a radioimmunoassay, surface plasmon resonance, or kinetic exclusion assay. In some embodiments, the recombinant proteins described herein, which bind to a human antigen, will bind to another species of the antigen protein with less than 10%, 15%, or 20% of the binding of the antibody to the human antigen protein as measured by, e.g., a radioimmunoassay, surface plasmon resonance, or kinetic exclusion assay.

As used herein, the term “host cell” can be any type of cell, e.g., a primary cell, a cell in culture, or a cell from a cell line. In embodiments, the term “host cell” refers to a cell transfected with a nucleic acid molecule and the progeny or potential progeny of such a cell. Progeny of such a cell cannot be identical to the parent cell transfected with the nucleic acid molecule, e.g., due to mutations or environmental influences that can occur in succeeding generations or integration of the nucleic acid molecule into the host cell genome.

In certain aspects, a recombinant protein described herein can be described by its VL domain alone, or its VH domain alone, or by its 3 VL CDRs alone, or its 3 VH CDRs alone. See, e.g., Rader C et al., (1998) PNAS 95:8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352:624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, provided herein are recombinant proteins that specifically bind to serum albumin (e.g., human serum albumin) and comprise the Chothia VL CDRs of a VL. In certain aspects, provided herein are antibodies that specifically bind to serum albumin (e.g., human serum albumin) and comprise the Chothia VH CDRs of a VH. In certain aspects, provided herein are antibodies that specifically bind to serum albumin (e.g., human serum albumin) and comprise the Chothia VL CDRs of a VL and comprise the Chothia VH CDRs of a VH. In certain embodiments, antibodies that specifically bind to serum albumin (e.g., human serum albumin) comprise one or more CDRs, in which the Chothia and Kabat CDRs have the same amino acid sequence. In certain embodiments, provided herein are antibodies that specifically bind to serum albumin and comprise combinations of Kabat CDRs and Chothia CDRs.

In certain aspects, the CDRs of an antibody can be determined according to the AbM numbering scheme, which refers AbM hypervariable regions that represent a compromise between the Kabat CDRs and Chothia structural loops and are used by Oxford Molecular's AbM antibody modeling software (Oxford Molecular Group, Inc.).

In some embodiments, the position of one or more CDRs along the VH (e.g., CDR1, CDR2, or CDR3) and/or VL (e.g., CDR1, CDR2, or CDR3) region of an antibody described herein can vary by one, two, three, four, five, or six amino acid positions so long as immunospecific binding to an antigen is maintained (e.g., substantially maintained, e.g., at least 50%, at least 60%, at least 70%, at least 80%, at least 90%, at least 95%). For example, the position defining a CDR of an antibody described herein can vary by shifting the N-terminal and/or C-terminal boundary of the CDR by one, two, three, four, five, or six amino acids, relative to the CDR position of an antibody described herein, so long as immunospecific binding to the antigen(s) is maintained (e.g., substantially maintained, e.g., at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of an antibody described herein can vary (e.g., be shorter or longer) by one, two, three, four, five, or more amino acids, so long as immunospecific binding to the antigen(s) is maintained (e.g., substantially maintained, e.g., at least 50%, at least 60%, at least 70%, at least 80%, at least 90%, at least 95%).

In some embodiments, a VL CDR1, VL CDR2, VL CDR3, VH CDR1, VH CDR2, and/or VH CDR3 described herein can be one, two, three, four, five or more amino acids shorter than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, a VL CDR1, VL CDR2, VL CDR3, VH CDR1, VH CDR2, and/or VH CDR3 described herein can be one, two, three, four, five or more amino acids longer than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the carboxy terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In some embodiments, the carboxy terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). Any method known in the art can be used to ascertain whether immunospecific binding to the antigen(s) is maintained, e.g., the binding assays and conditions described in the “Examples” section herein.

The percent identity between two sequences can be determined using techniques similar to those described above, with or without allowing gaps. In calculating percent identity, typically only exact matches are counted.

The recombinant proteins disclosed herein can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies can be used to detect antigen proteins.

### Polynucleotides, Vectors, and Cells

According to some aspects of the disclosure, provided are a nucleic acid encoding the recombinant fusion protein, an expression vector including the nucleic acid, and a cell transformed with the expression vector.

Since the nucleic acid, the expression vector, and the transformed cell include or use the aforementioned recombinant fusion protein or a nucleic acid encoding the recombinant fusion protein as it is, descriptions of common contents therebetween will be omitted.

For example, a recombinant fusion protein can be produced by separating the nucleic acid encoding the recombinant fusion protein. Following the separation of the nucleic acid, the resultant nucleic acid can be inserted into a replicable vector to be further cloned (i.e., amplification of DNA) or to be further expressed. In this regard, some aspects provide a vector including the nucleic acid.

The term “nucleic acid” as used in the present specification comprehensively includes DNA (gDNA and cDNA) and RNA molecules, and the term “nucleotide”, which is the basic structural unit in a nucleic acid, includes not only natural nucleotides, but also analogs in which sugar or base sites are modified.

Nucleic acid is also construed to include a nucleotide sequence exhibiting substantial identity to the nucleotide sequence. The substantial identity refers to nucleotide sequences exhibiting at least 80% homology, more preferably at least 90% homology, and most preferably at least 95% homology, in the case where the nucleotide sequences of the present disclosure and other sequences are aligned so as to correspond as much as possible, and the aligned sequences are analyzed by using algorithms commonly used in the art.

DNA encoding the recombinant fusion protein can be readily separated or synthesized by using conventional procedures (e.g., by using an oligonucleotide probe capable of binding specifically to DNA encoding the recombinant fusion protein). Many vectors are available. Vector components generally include, but are not limited to, one or more of the following: a signal sequence, an origin of replication, one or more marker genes, an enhancer element, a promoter, and a transcription termination sequence.

The term “vector” as used in the present specification refers to a plasmid vector as a means for expressing a target gene in a host cell, and examples thereof include: a plasmid vector; a cosmid vector; a viral vector such as a bacteriophage vector, an adenoviral vector, a retroviral vector, and an adeno-associated viral vector and the like. In the vector, the nucleic acid encoding the recombinant fusion protein can operably be linked to a promoter.

The expression “operably linked” as used in the present specification refers to a functional linkage between a control sequence of nucleic acid expression (e.g., a promoter, a signal sequence, or an array at a binding site for a transcriptional regulator) and another nucleic acid sequence, whereby the control sequence regulates transcription and/or translation of the other nucleic acid sequence.

In some embodiments, when a prokaryotic cell is used as a host, a strong promoter capable of initiating transcription (e.g., tac promoter, lac promoter, lacUV5 promoter, lpp promoter, pLλ promoter, pRλ promoter, rac5 promoter, amp promoter, recA promoter, SP6 promoter, trp promoter, T7 promoter, etc.), a ribosome binding site for initiation of translation, and a transcription/translation termination sequence are typically included. In one or more embodiments, when a eukaryotic cell is used as a host, a promoter derived from the genome of mammalian cells (e.g., metallothionein promoter, β-actin promoter, human hemoglobin promoter, and human muscle creatine promoter) or a promoter derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), and promoter of Rouss sarcoma virus (RSV)) can be used, and these promoters generally have a polyadenylation sequence as a transcription termination sequence. In one or more embodiments, the vector can be fused with other sequences to facilitate purification of the recombinant fusion proteins expressed from the vector. Sequences to be fused can include, for example, glutathione S-transferase (Pharmacia, USA), maltose-binding protein (NEB, USA), FLAG (IBI, USA), and 6×His (hexahistidine; Qiagen, USA), and the like. The vector can include, as a selectable marker, antibiotic resistance genes commonly used in the art, and for example, genes resistant to ampicillin, gentamicin, carbenicillin, chloramphenicol, streptomycin, kanamycin, geneticin, neomycin, and tetracycline can be included.

Some aspects of the disclosure provides a cell transformed with the aforementioned vector. Cells used to produce the recombinant fusion protein of the disclosure can be prokaryotic cells, yeast cells, or higher eukaryotic cells, but embodiments are not particularly limited thereto. For use as prokaryotic host cells, Escherichia coli, strains of the genus Bacillus, such as B. subtilis and B. thuringiensis, the genus Streptomyces, the genus Pseudomonas (e.g., P. putida), Proteus mirabilis, and the genus Staphylococcus (e.g., S. carnosus) can be used. However, animal cells are of greatest interest, and examples of useful host cell lines are COS-7, BHK, CHO (GS null CHO-K1), CHOK1, DXB-11, DG-44, CHO/-DHFR, CV1, COS-7, HEK293, BHK, TM4, VERO, HELA, MDCK, BRL 3A, WI-38, Hep G2, SK-Hep, MMT, TRI, MRC 5, FS4, 3T3, RIN, A549, PC12, K562, PER.C6, SP2/0, NS-0, U20S, or HT1080, but embodiments are not particularly limited thereto.

The term “transformation” as used in the present specification refers to a molecular biology technology in which a DNA chain fragment or a plasmid having foreign genes of a different kind from those of the original cell penetrates between cells and binds to the DNA existing in the original cell, thereby transforming the genetic traits of the original cell. The transformation can refer that an expression vector including the recombinant fusion protein gene is inserted into a host cell.

Disclosed herein are nucleic acid molecules encoding the recombinant proteins disclosed herein.

Disclosed herein are expression vectors comprising the nucleic acid molecules disclosed herein.

Disclosed herein are cells transformed with the expression vectors disclosed herein.

Since the nucleic acid, the expression vector, and the transformed cell include the above-described recombinant protein or the nucleic acid encoding the recombinant protein as it is, or they use the same, descriptions common thereto will be omitted.

For example, in some aspects, the recombinant protein can be produced by isolating the nucleic acid encoding the recombinant protein. The nucleic acid is isolated and inserted into a replicable vector to perform additional cloning (DNA amplification) or additional expression. On the basis of this, other aspects relate to a vector including the nucleic acid.

As used herein, the term “nucleic acid” or “nucleic acid molecule” comprehensively includes DNA (gDNA and cDNA) and RNA molecules, and nucleotides as basic units of the nucleic acid include not only natural nucleotides but also analogues having modified sugar or base moieties.

The nucleic acid is interpreted to include a nucleotide sequence showing substantial identity to the nucleotide sequence. Substantial identity means a nucleotide sequence showing at least 80% homology, more specifically at least 90% homology, and most specifically at least 95% homology, when the nucleotide sequence of the present disclosure and another optional sequence are aligned to correspond to each other as much as possible and the aligned sequences are analyzed using an algorithm commonly used in the art.

DNA encoding the recombinant protein is easily isolated or synthesized by using a common process (e.g., by using an oligonucleotide probe capable of specifically binding to the DNA encoding the recombinant protein). Many vectors are available. Vector components generally include, but are not limited to, one or more of the following: a signal sequence, an origin of replication, one or more marker genes, an enhancer element, a promoter, and a transcription termination sequence.

As used herein, the term “vector” includes, as a means to express a target gene in a host cell, plasmid vectors; cosmid vectors; viral vectors such as bacteriophage vectors, adenovirus vectors, retrovirus vectors, and adeno-associated virus vectors, etc. In the vector, the nucleic acid encoding the recombinant protein is operably linked to a promoter.

“Operably linked” refers to a functional linkage between a nucleic acid expression control sequence (e.g., a promoter, a signal sequence, an array of transcriptional regulatory factor binding sites) and another nucleic acid sequence, whereby the control sequence directs transcription and/or translation of another nucleic acid sequence.

When a prokaryotic cell is used as a host, a powerful promoter capable of directing transcription (e.g., tac promoter, lac promoter, lacUV5 promoter, lpp promoter, pLλ promoter, pRλ promoter, rac5 promoter, amp promoter, recA promoter, SP6 promoter, trp promoter and T7 promoter, etc.), a ribosome binding site for initiation of translation, and a transcription/translation termination sequence are generally included. For example, when a eukaryotic cell is used as a host, a promoter derived from the genome of a mammalian cell (e.g., metallothionein promoter, β-actin promoter, human hemoglobin promoter, and human muscle creatine promoter) or a promoter derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), and promoter of Rous sarcoma virus (RSV)) can be used, and a polyadenylated sequence can be commonly used as the transcription termination sequence. In some cases, the vector can be fused with another sequence to facilitate purification of the recombinant protein expressed therefrom. The sequence to be fused includes, e.g., glutathione S-transferase (Pharmacia, USA), maltose binding protein (NEB, USA), FLAG (IBI, USA), 6×His (hexahistidine; Quiagen, USA), etc. The vector includes, as a selective marker, an antibiotic-resistant gene that is ordinarily used in the art, e.g., genes resistant against ampicillin, gentamycin, carbenicillin, chloramphenicol, streptomycin, kanamycin, geneticin, neomycin, and tetracycline.

In still other aspects, the present disclosure provides cells transformed with the above-mentioned vectors. The cells used to produce the recombinant protein of the present disclosure can be prokaryotic cells, yeast cells, or higher eukaryotic cells, but are not limited thereto. Prokaryotic host cells such as Escherichia coli, the genus Bacillus strains such as Bacillus subtilis and Bacillus thuringiensis, Streptomyces, Pseudomonas (e.g., Pseudomonas putida), Proteus mirabilis and Staphylococcus (e.g., Staphylococcus carnosus) can be used. However, animal cells are most interested, and examples of the useful host cell line can include COS-7, BHK, CHO (GS null CHO-K1), CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but are not limited thereto.

As used herein, the term “transformation” means a molecular biological technique that changes the genetic trait of a cell by a DNA chain fragment or plasmid which possesses a different type of foreign gene from that of the original cell, penetrates among the cells, and combines with DNA in the original cell. The transformation means insertion of the expression vector including the gene of the recombinant protein into a host cell.

Provided herein are nucleic acid molecules comprising a nucleotide sequence encoding a recombinant protein described herein (e.g., a variable light chain region and/or variable heavy chain region) that immunospecifically binds to an antigen, and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In some embodiments, a nucleic acid molecule(s) encoding an antibody described herein is isolated or purified.

Provided herein are polynucleotides comprising nucleotide sequences encoding antibodies, which immunospecifically bind to an antigen polypeptide (e.g., human serum albumin) and comprises an amino acid sequence as described herein, as well as antibodies that compete with such antibodies for binding to an antigen polypeptide (e.g., in a dose-dependent manner), or which binds to the same epitope as that of such antibodies.

Provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein. The polynucleotides can comprise nucleotide sequences encoding a heavy chain comprising the VH FRs and CDRs of antibodies described herein.

Provided herein are polynucleotides comprising a nucleotide sequence encoding a recombinant protein comprising a Fab comprising three VH chain CDRs, e.g., containing VL CDR1, VL CDR2, and VL CDR3 of an antibody to human serum albumin described herein and three VH chain CDRs, e.g., containing VH CDR1, VH CDR2, and VH CDR3 of an antibody to human serum albumin described herein.

Provided herein are polynucleotides comprising a nucleotide sequence encoding a recombinant protein comprising a VL domain.

In certain embodiments, a polynucleotide described herein comprises a nucleotide sequence encoding a recombinant protein provided herein comprising a light chain variable region comprising an amino acid sequence described herein (e.g., SEQ ID NO:61, 62, 63, 64, 65, 66, or 67), wherein the antibody immunospecifically binds to serum albumin.

In certain embodiments, a polynucleotide described herein comprises a nucleotide sequence encoding an antibody provided herein comprising a heavy chain variable region comprising an amino acid sequence described herein (e.g., SEQ ID NO:55, 56, 57, 58, 59, or 60), wherein the antibody immunospecifically binds to serum albumin.

In specific aspects, provided herein are polynucleotides comprising a nucleotide sequence encoding an antibody comprising a light chain and a heavy chain, e.g., a separate light chain and heavy chain. With respect to the light chain, in some embodiments, a polynucleotide provided herein comprises a nucleotide sequence encoding a kappa light chain. In other embodiments, a polynucleotide provided herein comprises a nucleotide sequence encoding a lambda light chain. In yet other embodiments, a polynucleotide provided herein comprises a nucleotide sequence encoding an antibody described herein comprising a human kappa light chain or a human lambda light chain. In some embodiments, a polynucleotide provided herein comprises a nucleotide sequence encoding an antibody, which immunospecifically binds to serum albumin, wherein the antibody comprises a light chain, and wherein the amino acid sequence of the VL domain can comprise the amino acid sequence set forth in SEQ ID NO:61, 62, 63, 64, 65, 66, or 67 and wherein the constant region of the light chain comprises the amino acid sequence of a kappa light chain constant region.

Also provided herein are polynucleotides encoding an antibody or a fragment thereof that are optimized, e.g., by codon/RNA optimization, replacement with heterologous signal sequences, and elimination of mRNA instability elements. Methods to generate optimized nucleic acids encoding an antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an antibody described herein or a fragment thereof (e.g., VL domain or VH domain) can hybridize to an antisense (e.g., complementary) polynucleotide of an unoptimized polynucleotide sequence encoding an antibody described herein or a fragment thereof (e.g., VL domain or VH domain). In specific embodiments, an optimized nucleotide sequence encoding an antibody described herein or a fragment hybridizes under high stringency conditions to antisense polynucleotide of an unoptimized polynucleotide sequence encoding an antibody described herein or a fragment thereof. In some embodiments, an optimized nucleotide sequence encoding an antibody described herein or a fragment thereof hybridizes under high stringency, intermediate or lower stringency hybridization conditions to an antisense polynucleotide of an unoptimized nucleotide sequence encoding an antibody described herein or a fragment thereof. Information regarding hybridization conditions has been described, see, e.g., US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17:242-246), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or fragment thereof described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, e.g., to generate chimeric and humanized antibodies.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, such as poly A+ RNA, isolated from, any tissue or cells expressing the antibody, such as hybridoma cells selected to express an antibody described herein) by PCR amplification using synthetic primers capable of hybridizing to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding recombinant proteins described herein can be readily isolated and sequenced using conventional procedures (e.g., by using oligonucleotide probes that are capable of binding specifically to genes encoding the heavy and light chains of the recombinant proteins). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of recombinant proteins in the recombinant host cells.

To generate antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, e.g., by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6×sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridizations under other stringent hybridization conditions are known to those of skill in the art and have been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3.

Other aspects provide recombinant vectors comprising the gene encoding the IL-2m and the nucleic acid encoding the antigen binding fragment against serum albumin. Still other aspects provide a cell transformed with the vector.

Disclosed herein are nucleic acid molecules encoding a heavy chain region comprising an amino acid sequence having at least 90%, at least 93%, at least 95%, at least 96%, at least 97%, at least 98%, at least 99%, or 100% identity to SEQ ID NO:6. Disclosed herein are nucleic acid molecules encoding a heavy chain region comprising a nucleotide sequence having at least 90%, at least 93%, at least 95%, at least 96%, at least 97%, at least 98%, at least 99%, or 100% identity to SEQ ID NO:7 or 8.

Disclosed herein are nucleic acid molecules encoding a light chain region comprising an amino acid sequence having at least 90%, at least 93%, at least 95%, at least 96%, at least 97%, at least 98%, at least 99%, or 100% identity to SEQ ID NO:13. Disclosed herein are nucleic acid molecules encoding a light chain region comprising a nucleotide sequence having at least 90%, at least 93%, at least 95%, at least 96%, at least 97%, at least 98%, at least 99%, or 100% identity to SEQ ID NO:14 or 15.

In some embodiments, disclosed herein are nucleic acids, each encoding the heavy chain region of SEQ ID NO:6 and the light chain region of SEQ ID NO:13. In some embodiments, the nucleic acid encoding the heavy chain region of SEQ ID NO:6 can be represented by SEQ ID NO:7 or SEQ ID NO:8, and the nucleic acid encoding the light chain region of SEQ ID NO:13 can be represented by SEQ ID NO:14 or SEQ ID NO:15.

Further disclosed herein are expression vectors comprising:
(a) a promoter,
(b) a first nucleic acid molecule encoding a light chain that binds to serum albumin, and
(c) a second nucleic acid molecule encoding heavy chain and a bioactive effector moiety such as IL-2m and a linker,
wherein the promoter, the first nucleic acid sequence, and the second nucleic acid molecules are operably linked. The second nucleic acid molecule can encode 1, 2, 3, 4, 5, 6, or more bioactive effector moieties and linkers.

Also disclosed herein are expression vectors comprising:
(a) a promoter and
(b) a nucleic acid molecule encoding a heavy chain variable domain as disclosed herein and a heavy chain constant domain as disclosed herein.

Also disclosed herein are expression vectors comprising:
(a) a promoter and
(b) a nucleic acid molecule encoding a IL18BP as disclosed herein, a heavy chain variable domain as disclosed herein, and a heavy chain constant domain as disclosed herein.

Also disclosed herein are expression vectors comprising:
(a) a promoter and
(b) a nucleic acid molecule encoding a light chain variable domain as disclosed herein and a light chain constant domain as disclosed herein.

Also disclosed herein are expression vectors comprising:
(a) a promoter and
(b) a nucleic acid molecule encoding an IL-2m as disclosed herein, a light chain variable domain as disclosed herein, and a light chain constant domain as disclosed herein. One, two, three, or more expression vectors or nucleic acid molecules can be expressed to produce the desired recombinant proteins.

In some embodiments, a first nucleic acid molecule or vector comprises a nucleic acid sequence encoding a recombinant protein comprising an antigen binding fragment comprising a heavy chain, wherein the heavy chain comprises a heavy chain variable domain and a heavy chain constant domain, wherein the heavy chain variable domain comprises
(1) a heavy chain complementarity determining domain 1 (CDR1) comprising the amino acid sequence of SYGIS (SEQ ID NO:22),
   a heavy chain complementarity determining domain 2 (CDR2) comprising the amino acid sequence of WINTYSGGTKYAQKFQG (SEQ ID NO:23), and
   a heavy chain complementarity determining domain 3 (CDR3) comprising the amino acid sequence of LGHCQRGICSDALDT (SEQ ID NO:24);
(2) a heavy chain CDR1 comprising the amino acid sequence of SYGIS (SEQ ID NO: 22),
   a heavy chain CDR2 comprising the amino acid sequence of RINTYNGNTGYAQRLOG (SEQ ID NO:25), and
   a heavy chain CDR3 comprising the amino acid sequence of LGHCQRGICSDALDT (SEQ ID NO:24);
(3) a heavy chain CDR1 comprising the amino acid sequence of NYGIH (SEQ ID NO: 26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
(4) a heavy chain CDR1 comprising the amino acid sequence of SYAMS (SEQ ID NO: 29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
(5) a heavy chain CDR1 comprising the amino acid sequence of AYWIA (SEQ ID NO: 32),
   a heavy chain CDR2 comprising the amino acid sequence of MIWPPDADARYSPSFOG (SEQ ID NO:33), and
   a heavy chain CDR3 comprising the amino acid sequence of LYSGSYSP (SEQ ID NO: 34); or
(6) a heavy chain CDR1 comprising the amino acid sequence of AYSMN (SEQ ID NO: 35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37).

Disclosed herein is a second nucleic acid molecule or vector comprises a nucleic acid sequence encoding a recombinant protein comprising an antigen binding fragment comprising a light chain, wherein the light chain comprises a light chain variable domain and a light chain constant domain, wherein the light chain variable domain comprises
(7) a light chain CDR1 comprising the amino acid sequence of RASQSISRYLN (SEQ ID NO:38),
   a light chain CDR2 comprising the amino acid sequence of GASRLES (SEQ ID NO: 39), and
   a light chain CDR3 comprising the amino acid sequence of QQSDSVPVT (SEQ ID NO:40);
(8) a light chain CDR1 comprising the amino acid sequence of RASQSISSYLN (SEQ ID NO:41),
   a light chain CDR2 comprising the amino acid sequence of AASSLQS (SEQ ID NO: 42), and
   a light chain CDR3 comprising the amino acid sequence of QQSYSTPPYT (SEQ ID NO:43);
(9) a light chain CDR1 comprising the amino acid sequence of RASQSIFNYVA (SEQ ID NO:44),
   a light chain CDR2 comprising the amino acid sequence of DASNRAT (SEQ ID NO: 45), and
   a light chain CDR3 comprising the amino acid sequence of QQRSKWPPTWT (SEQ ID NO:46);
(10) a light chain CDR1 comprising the amino acid sequence of RASETVSSRQLA (SEQ ID NO:47),
   a light chain CDR2 comprising the amino acid sequence of GASSRAT (SEQ ID NO: 48), and
   a light chain CDR3 comprising the amino acid sequence of QQYGSSPRT (SEQ ID NO:49);
(11) a light chain CDR1 comprising the amino acid sequence of RASQSVSSSSLA (SEQ ID NO:50),
   a light chain CDR2 comprising the amino acid sequence of GASSRAT (SEQ ID NO: 48), and
   a light chain CDR3 comprising the amino acid sequence of QKYSSYPLT (SEQ ID NO: 51); or
(12) a light chain CDR1 comprising the amino acid sequence of RASQSVGSNLA (SEQ ID NO:52),
   a light chain CDR2 comprising the amino acid sequence of GASTGAT (SEQ ID NO: 53), and
   a light chain CDR3 comprising the amino acid sequence of QQYYSFLAKT (SEQ ID NO:54).

For example, the nucleic acid molecule encoding IL-2m can be linked to the first or second nucleic acid molecule or vector described above.

In other embodiments, the first nucleic acid molecule can comprise a nucleic acid sequence encoding a Fab comprising: a heavy chain variable domain comprising (1) above and a light chain variable domain comprising (7) above; a heavy chain variable domain comprising (2) above and a light chain variable domain comprising (8) above; a heavy chain variable domain comprising (3) above and a light chain variable domain comprising (9) above; a heavy chain variable domain comprising (4) above and a light chain variable domain comprising (10) above; a heavy chain variable domain comprising (5) above and a light chain variable domain comprising (11) above; a heavy chain variable domain comprising (6) above and a light chain variable domain comprising (12) above; or any or all combinations of a heavy chain variable domain and a light chain variable domain described above. In some embodiments, the first nucleic acid molecule comprises a nucleic acid sequence encoding a Fab (SL335) comprising the heavy chain variable domain comprises a heavy chain CDR1 comprising the amino acid sequence of SEQ ID NO:35, a heavy chain CDR2 comprising the amino acid sequence of SEQ ID NO: 36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO: 53, and a light chain CDR3 comprising the amino acid sequence of SEQ ID NO:54. The first or second nucleic acid molecule can encode the IL-2m.

In other embodiments, a first nucleic acid molecule or vector comprises a nucleic acid sequence encoding a Fab comprising a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60. In some embodiments, a second nucleic acid molecule or vector comprises a nucleic acid sequence encoding a Fab comprising a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, 66, or 67. The nucleic acid molecule encoding IL-2m can be linked to the first or second nucleic acid molecule or vector.

In some embodiments, a first nucleic acid molecule or vector comprises a nucleic acid sequence encoding a Fab comprising a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60, and a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, or 66 or 67, respectively.

In some embodiments, the first nucleic acid molecule comprises a nucleic acid sequence encoding a Fab (SL335) comprising a heavy chain domain comprising an amino acid sequence having at least 90%, at least 93%, at least 95%, at least 96%, at least 97%, at least 98%, at least 99%, or 100% identity to SEQ ID NO:6 (V_{H}-C_{HI} domain) and a light chain domain comprising an amino acid sequence having at least 90%, at least 93%, at least 95%, at least 96%, at least 97%, at least 98%, at least 99%, or 100% identity to SEQ ID NO:13 (V_{L}-C_{κ} domain).

In some embodiments, the bioactive effector moiety is IL-2m. In some embodiments, a nucleic acid molecule encodes an IL-2m protein comprises an amino acid sequence having at least 90%, at least 93%, at least 95%, at least 96%, at least 97%, at least 98%, at least 99%, or 100% identity to SEQ ID NO:1 or 2.

Recombinant expression of an antibody or fragment thereof described herein (e.g., a heavy or light chain of an antibody described herein) that specifically binds to involves construction of an expression vector containing a polynucleotide that encodes the antibody or fragment. Once a polynucleotide encoding an antibody or fragment thereof (e.g., heavy or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, e.g., in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, e.g., include the nucleotide sequence encoding the constant region of the antibody molecule (see, e.g., WO86/05807 and WO89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein.

A variety of host-expression vector systems can be utilized to express antibody molecules described. Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, cells for expressing antibodies described herein (e.g., an antibody comprising the CDRs of any one of antibodies pab1949 or pab2044) are CHO cells, e.g., CHO cells from the CHO GS System™ (Lonza). In some embodiments, cells for expressing antibodies described herein are human cells, e.g., human cell lines. In some embodiments, a mammalian expression vector is pOptiVEC™ or pcDNA3.3. In some embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In certain embodiments, antibodies described herein are produced by CHO cells or NS0 cells. In some embodiments, the expression of nucleotide sequences encoding antibodies described herein is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794), in which the antibody coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan J & Shenk T (1984) PNAS 81:3655-3659). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol 153:516-544).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, recombinant proteins described herein (e.g., an antibody comprising the CDRs are produced in mammalian cells, such as CHO cells.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an antibody described herein or a fragment thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11 (1): 223-232), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-823) genes can be employed in tk-, hgprt- or aprt-cells, respectively. Also, antimetabolite resistance can be used as the basis of selection for the following genes: dhfr, which confers resistance to methotrexate (Wigler M et al., (1980) PNAS 77 (6): 3567-3570; O'Hare K et al., (1981) PNAS 78:1527-1531); gpt, which confers resistance to mycophenolic acid (Mulligan R C & Berg P (1981) PNAS 78 (4): 2072-2076); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11 (5): 211-215); and hygro, which confers resistance to hygromycin (Santerre R F et al., (1984) Gene 30 (1-3): 147-156).

Once an antibody molecule described herein has been produced by recombinant expression, it can be purified by any method known in the art for purification of an immunoglobulin molecule, e.g., by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in some embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, e.g., different post-translational modified forms of an antibody. When the antibody or fragment is recombinantly produced, it is also generally substantially free of culture medium, i.e., culture medium represents less than about 20%, 10%, 2%, 1%, 0.5%, or 0.1% of the volume of the protein preparation. When the antibody or fragment is produced by chemical synthesis, it is generally substantially free of chemical precursors or other chemicals, i.e., it is separated from chemical precursors or other chemicals which are involved in the synthesis of the protein. Accordingly, such preparations of the antibody or fragment have less than about 30%, 20%, 10%, or 5% (by dry weight) of chemical precursors or compounds other than the antibody or fragment of interest. In some embodiments, antibodies described herein are isolated or purified.

### Antibody Production

Still other aspects provide methods of preparing the recombinant protein, the methods including (a) culturing the cells; and (b) recovering the recombinant protein from the cultured cells. The cells can be cultured in various media. A commercially available medium can be used as a culture medium without limitation. All other essential supplements known to those skilled in the art can also be included at appropriate concentrations. Culture conditions, e.g., temperature, pH, etc., are those previously used together with the host cell selected for expression, and will be apparent to those skilled in the art. The recovering of the recombinant proteins can be performed by removing impurities by, e.g., centrifugation or ultrafiltration, and purifying the resultant by, e.g., affinity chromatography, etc. Other additional purification techniques, e.g., anion or cation exchange chromatography, hydrophobic interaction chromatography, hydroxylapatite chromatography, etc. can be used.

Recombinant proteins disclosed herein can be produced by any method known in the art for the synthesis of antibodies, e.g., by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, e.g., in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some embodiments, the recombinant proteins described herein are antibodies (e.g., recombinant antibodies) prepared, expressed, created, or isolated by any means that involves creation, e.g., via synthesis or genetic engineering of DNA sequences. In certain embodiments, such antibodies comprise sequences (e.g., DNA sequences or amino acid sequences) that do not naturally exist within the antibody germline repertoire of an animal or mammal (e.g., human) in vivo.

In some aspects, provided herein are methods of making recombinant proteins disclosed herein comprising culturing a cell or host cell as described herein. In some aspects, provided herein are methods of making a recombinant protein comprising expressing (e.g., recombinantly expressing) the antibodies using a cell or host cell described herein (e.g., a cell or a host cell comprising polynucleotides encoding an antibody described herein). In some embodiments, the cell is an isolated cell. In some embodiments, the exogenous polynucleotides has been introduced into the cell. In some embodiments, the method further comprises purifying the antibody obtained from the cell or host cell.

Antibodies can be prepared using a wide variety of techniques known in the art including the use of hybridoma, recombinant, and phage display technologies, or a combination thereof. For example, monoclonal antibodies can be produced using hybridoma techniques including those known in the art and taught, e.g., in Harlow E & Lane D, Antibodies: A Laboratory Manual (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein.

A “monoclonal antibody,” as used herein, is an antibody produced by a single cell (e.g., hybridoma or host cell producing a recombinant antibody), wherein the antibody immunospecifically binds to an antigen (e.g., human serum albumin) as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody can be a Fab fragment or a F(ab′)₂ fragment. Monoclonal antibodies described herein can, e.g., be made by the hybridoma method as described in Kohler G & Milstein C (1975) Nature 256:495 or can, e.g., be isolated from phage libraries using the techniques as described herein, for example. Other methods for the preparation of clonal cell lines and of monoclonal antibodies expressed thereby are well known in the art (see, e.g., Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the antigen (e.g., human serum albumin)) used for immunization. Alternatively, lymphocytes can be immunized in vitro. Lymphocytes then are fused with myeloma cells using a suitable fusing agent, such as polyethylene glycol, to form a hybridoma cell. Some aspects of the disclosure provide a method of preparing the recombinant fusion protein, the method including: (a) culturing the cell; and (b) recovering the recombinant fusion protein from the cultured cell. The cell can be cultured in various media.

For use as a culture medium, any commercially available medium can be used without limitation. All other necessary supplements known to those skilled in the art can also be included in suitable concentrations. Culture conditions, such as temperature, pH, and the like, have already been applied to host cells selected for expression, and will be apparent to those skilled in the art. The recombinant fusion protein can be purified by, for example, centrifugation or ultrafiltration to remove impurities, and the resultant product can be purified by using, for example, affinity chromatography or the like. Other purification techniques, such as anion or cation exchange chromatography, hydrophobic interaction chromatography, hydroxyapatite chromatography, and the like, can be used.

### Compositions and Uses Thereof

Disclosed herein are compositions comprising the recombinant fusion proteins disclosed herein and a carrier. Also disclosed herein are pharmaceutical compositions comprising the recombinant fusion proteins disclosed herein and pharmaceutically acceptable carriers.

Disclosed herein are methods of treating for preventing or treating autoimmune disease or inflammatory neurological disease in a subject in need thereof, comprising administering the pharmaceutical compositions disclosed herein to the subject. Also disclosed herein are uses of pharmaceutical compositions for preventing or treating autoimmune disease or inflammatory neurological disease, comprising the recombinant fusion protein disclosed herein as an active ingredient. In some embodiments, the autoimmune disease is selected from the group consisting of Crohn's disease, ulcerative colitis, spondylitis ankylopoietica, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, restenosis of a blood vessel, glomerulonephritis, and gastrointestinal allergy, and the inflammatory neurological disease is selected from the group consisting of amyotrophic lateral sclerosis and Parkinson's disease.

Provided are pharmaceutical compositions for preventing or treating an immune disease, including the recombinant fusion protein as an active ingredient. Provided is a method of preventing or treating an immune disease, including administering the recombinant fusion protein to a subject. Details of the recombinant fusion protein are as described above.

The immune disease can be inflammatory bowel disease, systemic lupus erythematosus, amyotrophic lateral sclerosis, or Parkinson's disease.

In some embodiments, the pharmaceutical compositions can include the recombinant fusion protein for preventing or treating inflammatory bowel disease. Inflammatory bowel disease is a representative autoimmune disease that causes abdominal pain, diarrhea, and bleeding due to a severe chronic inflammatory response, and is classified into Chron's disease and ulcerative colitis. Chron's disease causes random inflammation throughout the digestive tract, whereas ulcerative colitis causes inflammation confined to the large intestine. The exact pathogenesis is not yet known, and exacerbation and alleviation of the disease are known to be repeated without a clear treatment method. Although it has been confirmed that increasing the number of regulatory T cells has a disease suppression effect in animal models of various autoimmune diseases, there is currently no approved therapeutic agent that increases the number of regulatory T cells in vivo. Therefore, the recombinant fusion protein according to some aspects can be effectively used for the treatment of inflammatory bowel disease.

Provided are pharmaceutical compositions for preventing or treating systemic lupus, amyotrophic lateral sclerosis, or Parkinson's disease, comprising the recombinant fusion protein as an active ingredient.

In some embodiments, the pharmaceutical compositions can comprise (a) a pharmaceutically effective amount of the recombinant protein; and (b) a pharmaceutically acceptable carrier.

In some embodiments, the in vivo half-life of the pharmaceutical compositions can exhibit a 2- to 20-fold increase, as compared with that of human IL-2m. The in vivo half-life can exhibit, e.g., about 2.5-fold to about 3.5-fold, about 3.5-fold to about 6-fold increase, about 4-fold to about 6-fold increase, about 4.5-fold to about 6-fold increase, about 5-fold to about 6-fold increase, about 5.5-fold to about 6-fold increase, about 3-fold to about 5.5-fold increase, about 3.5-fold to about 5.5-fold increase, about 4-fold to about 5.5-fold increase, about 4.5-fold to about 5.5-fold increase, about 5-fold to about 5.5-fold increase, about 3-fold to about 5-fold increase, about 3.5-fold to about 5-fold increase, about 4-fold to about 5-fold increase, about 4.5-fold to about 5-fold increase, about 3-fold to about 4.5-fold increase, about 3.5-fold to about 4.5-fold increase, about 4-fold to about 4.5-fold increase, or any fold or ranges of folds derived therefrom, as compared with that of human IL-2m. In some embodiments, the in vivo half-life of the human IL-2m can be evaluated after subcutaneous injection of the human IL-2m.

In some embodiments, the pharmaceutical compositions can decrease white blood cell levels in blood. The white blood cells can be, e.g., neutrophils, monocytes, basophils, or a combination thereof. In some embodiments, the decreased white blood cell level can be sustained and maintained until day 20 after administration, until day 15 after administration, until day 12 after administration, until day 10 after administration, until day 8 after administration, until day 7 after administration, or any ranges derived therefrom.

The pharmaceutical compositions for preventing or treating an immune disease according to some aspects can be administered to mammals, such as rats, mice, livestock, humans, and the like, via various routes. All administration methods can be, for example, performed orally or rectally or by an intravenous, intramuscular, subcutaneous, intrauterine dural, or intracerebroventricular injection.

### Routes of Administration & Dosages

The pharmaceutical compositions of the present disclosure can be administered to a subject through a variety of administration routes including oral, transcutaneous, subcutaneous, intravenous, and intramuscular administration routes.

The amount of a recombinant protein or composition disclosed herein that will be effective in the treatment and/or prevention of a condition will depend on the nature of the disease and can be determined by standard clinical techniques.

In the present disclosure, the amount of the recombinant protein disclosed herein that is actually administered is determined in light of various relevant factors including the disease to be treated, a selected route of administration, the age, sex and body weight of a patient, and severity of the disease, and the type of a bioactive polypeptide as an active ingredient. Since the recombinant protein of the present disclosure has excellent sustainability in blood, the number and frequency of administration of the peptide preparations comprising the recombinant protein of the present disclosure can be noticeably reduced.

The pharmaceutical compositions are administered in a pharmaceutically effective amount. As used herein, the “pharmaceutically effective amount” or “effective amount” in the context of the administration of a therapy to a subject refers to the amount of a therapy that achieves a desired prophylactic or therapeutic effect. An effective dose level can be determined depending on factors including a patient's disease type, severity, drug activity, drug sensitivity, administration time, administration route and excretion ratio, treatment period, and co-administered drugs, and other factors well known in the medical field. The pharmaceutical compositions can be administered as a single therapeutic agent or in combination with other therapeutic drugs, and can be administered with existing therapeutic drugs simultaneously, separately, or sequentially, once or in a few divided doses. It is important to administer the composition in a minimum amount sufficient to obtain the maximum effect without any side effects, considering all the factors, and this amount can be easily determined by those skilled in the art. As used herein, the term “pharmaceutically effective amount” refers to an amount sufficient to treat or induce the disease or condition discussed herein, such as inducing superovulation or treating anovulation, hypogonadism, or polycystic ovary syndrome.

An appropriate dosage of the pharmaceutical compositions varies depending on a patient's conditions, body weight, disease severity, drug formulation, administration route and period, but can be appropriately selected by those skilled in the art. However, for desirable effects, the pharmaceutical compositions can be administered at a daily dose of 0.0001 mg/kg to 2,000 mg/kg, and specifically, 0.001 mg/kg to 2,000 mg/kg. Administration can be performed once a day, or in several divided doses.

The precise dose to be employed in the compositions will also depend on the route of administration, and the seriousness of the disease, and should be decided according to the judgment of the practitioner and each subject's circumstances. For example, effective doses can also vary depending upon means of administration, target site, physiological state of the patient (including age, body weight and health), other medications administered, or whether treatment is prophylactic or therapeutic. Usually, the patient is a human but can be a non-human, such as pets, e.g., dogs and cats. Treatment dosages are optimally titrated to optimize safety and efficacy.

In certain embodiments, an in vitro assay is employed to help identify optimal dosage ranges. Effective doses can be extrapolated from dose response curves derived from in vitro or animal model test systems.

In some embodiments, the recombinant fusion protein can be administered at a dose of 0.001 mg/kg to 2,000 mg/kg. For example, the recombinant fusion protein can be administered at a dose of 0.001 mg/kg to 0.01 mg/kg, 0.1 mg/kg to 1 mg/kg, 1.5 mg/kg to 2 mg/kg, 4 mg/kg to 10 mg/kg, 15 mg/kg to 20 mg/kg, 30 mg/kg to 40 mg/kg, 60 mg/kg to 80 mg/kg, 100 mg/kg to 200 mg/kg, or any dose or ranges of doses derived therefrom.

The pharmaceutical compositions can be prepared in a unit dosage form or in a multi-dose container by formulating using a pharmaceutically acceptable carrier and/or excipient according to a method that can be easily carried out by a person skilled in the art to which the present disclosure pertains. In this case, the formulation can be in the form of a solution, suspension, or emulsion in an oily or aqueous medium, or in the form of an extract, a suppository, a powder, granules, a tablet, or a capsule, and the formulation can further include a dispersing agent or a stabilizing agent.

Provided herein are compositions comprising a recombinant protein described herein having the desired degree of purity in a physiologically acceptable carrier, excipient or stabilizer (Remington's Pharmaceutical Sciences (1990) Mack Publishing Co., Easton, PA). Also disclosed herein are pharmaceutical compositions comprising a recombinant protein described herein and a pharmaceutically acceptable excipient. Acceptable carriers, excipients, or stabilizers are nontoxic to recipients at the dosages and concentrations employed. The pharmaceutical compositions for preventing or treating an immune disease according to some aspects can be formulated and used in the form of oral formulations, such as powders, granules, tablets, capsules, suspensions, emulsions, syrups, aerosols, and the like, external preparation formulations, suppositories, and sterile injection solution formulations, each prepared according to methods in the art. For formulation, a suitable carrier, excipient, or diluent commonly used in the preparation of the pharmaceutical compositions can be included.

For use as the carrier, excipient, and diluent, various compounds or mixtures including lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, or mineral oil can be used.

When forming into formulation, a commonly used diluent or excipient, such as a filler, an expander, a binder, a wetting agent, a disintegrant, a surfactant, and the like, can be used for preparation.

A solid formulation for oral administration can be prepared by mixing the recombinant fusion protein with at least one excipient, such as starch, calcium carbonate, sucrose, lactose, gelatin, and the like. Also, in addition to a simple excipient, a lubricant, such as magnesium stearate and talc, can be used.

For use as a liquid formulation for oral administration, a suspension, an oral liquid, an emulsion, a syrup, and the like can be used. In addition to water and liquid paraffin, which are simple diluents commonly used, various excipients, such as a wetting agent, a sweetening agent, a flavoring agent, a preservative, and the like can be included.

For use as a formulation for parenteral administration, a sterile solution, a non-aqueous agent, a suspension, an emulsion, a freeze-dried agent, a suppository, and the like can be included. Examples of the non-aqueous agent and the suspension are propylene glycol, polyethylene glycol, plant oil such as olive oil, and injectable ester such as ethyl oleate. For use as a base agent for the suppository, witepsol, macrogol, tween 61, cacao butter, laurin butter, glycerol, gelatin, or the like can be used.

The dosage of the pharmaceutical compositions for preventing or treating an immune disease according to some embodiments can vary depending on a condition of a patient, a weight of a patient, severity of a disease, a drug form, an administration route, and an administration period, but can be appropriately selected by those skilled in the art. However, for a desirable effect, the dosage can be in a range of about 0.0001 mg/kg to about 2,000 mg/kg per day, about 0.001 mg/kg to about 2,000 mg/kg per day. The administration can be performed once a day or several times a day.

The pharmaceutical compositions for treating diseases or inducing conditions can be administered to mammals, such as rats, mice, livestock, humans, etc., via various routes. All modes of administration can be contemplated, for example, by oral, rectal or intravenous, intramuscular, subcutaneous, or intradural administration, or intracerebroventricular injection.

The pharmaceutical compositions can be orally or parenterally administered. Specifically, the pharmaceutical compositions can be parenterally administered, and in this case, it can be administered by intravenous injection, subcutaneous injection, intramuscular injection, intraperitoneal injection, endothelial administration, topical administration, intranasal administration, intrapulmonary administration, and rectal administration. In some embodiments, it can be administered in the form of subcutaneous injection. When orally administered, a protein or peptide is digested, and therefore, it is required to formulate an oral composition by coating the active ingredient or protecting it from degradation in the stomach. In addition, the pharmaceutical compositions can be administered by any device capable of delivering an active substance to target cells.

Provided are health functional food compositions for preventing or treating an immune disease, including the recombinant fusion protein as an active ingredient. Provided are health functional food compositions for preventing or treating a cancer, including the recombinant fusion protein as an active ingredient. Details of the recombinant fusion protein, immune disease, and cancer are as described above.

Regarding the health functional food for preventing or ameliorating an immune disease or a cancer according to some aspects, when the recombinant fusion protein is used as an additive to the health functional food, the recombinant fusion protein can be added as it is or used in combination with other foods or food ingredients, appropriately according to methods known in the art. Here, a mixing amount of the active ingredient can be appropriately determined according to each purpose of use, such as prevention, health, or treatment.

The health functional food can be in any form of powders, granules, pills, tablets, or capsules, as well as a general food or beverage form.

Types of the general food are not particularly limited, and examples of the general food to which the substance can be added include meat, sausage, bread, chocolate, candy, snacks, confectionery, pizza, ramen, other noodles, gum, dairy products including ice cream, various soups, beverage, tea, drinks, alcoholic beverages, vitamin complex, and the like, and can include all foods in the ordinary sense.

In general, when preparing a food or a beverage, the recombinant fusion protein can be added in an amount of 15 parts by weight or less, 10 parts by weight or less, based on 100 parts by weight of the raw material. However, in the case of long-term intake for the purpose of health and hygiene or health control, the amount can be less than the ranges above. Also, since the present disclosure has no problem in terms of safety in that fractions from natural products are used, the amount greater than the ranges above can be used.

In the health functional food according to some aspects, a beverage can contain, as additive ingredients, various flavoring agents or native carbohydrates as in general beverages. Examples of the native carbohydrate can include monosaccharides, such as glucose and fructose, disaccharides, such as maltose and sucrose, polysaccharides, such as dextrin and cyclodextrin, and sugar alcohols, such as xylitol, sorbitol, and erythritol. For use as the sweetening agent, a natural sweetening agent, such as thaumatin and a stevia extract, a synthetic sweetening agent, such as saccharin, aspartame, and the like can be used. A proportion of the native carbohydrate in the beverage can be in a range of about 0.01 g to about 0.04 g, about 0.02 g to about 0.03 g, per 100 mL of the beverage according to the present disclosure.

Furthermore, the health functional food compositions for preventing or ameliorating an immune disease or a cancer according to some aspects can include various nutrients, vitamins, electrolytes, flavors, coloring agents, pectic acid and a salt thereof, alginic acid and a salt thereof, organic acids, protective colloidal thickeners, pH adjusters, stabilizers, preservatives, glycerin, alcohol, carbonizing agent used in carbonated beverages. Moreover, the compositions for sleep improvement of the present disclosure can include fruit pulps for preparing a natural fruit juice, a fruit juice beverage, and a vegetable juice. Such components can be used independently or in combination. A ratio of these additives is not limited but is generally selected from the range of about 0.01 parts by weight to about 0.1 parts by weight based on 100 parts by weight of the health functional food composition.

As described above, the recombinant fusion protein can exhibit, e.g., a half-life about 3.5 times extended in rats, as compared with the human recombinant IL-2m, and exhibits a biological activity at a similar level to that of IL-2m not fused to SL335. Therefore, since the recombinant fusion protein can exhibit similar efficacy even with less frequency of administration, patients can be administered with the drug at more convenient intervals.

### Kits

Disclosed herein are kits comprising the compositions or pharmaceutical compositions disclosed herein and labels comprising instructions for uses.

Provided herein are kits comprising one or more recombinant proteins described herein or conjugates thereof. Disclosed herein are kits comprising the compositions disclosed herein and labels comprising instructions for uses thereof. In some embodiments, provided herein is a pharmaceutical pack or kit comprising one or more containers filled with one or more of the ingredients of the pharmaceutical compositions described herein, such as one or more recombinant proteins provided herein. In some embodiments, the kits contain one or more pharmaceutical compositions described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration. Also provided herein are kits that can be used in the above methods. In some embodiments, a kit comprises a recombinant protein described herein, e.g., a purified recombinant protein, in one or more containers. In some embodiments, kits described herein contain a substantially isolated antigen(s) (e.g., human serum albumin) that can be used as a control. In other embodiments, the kits described herein further comprise a control antibody which does not react with a serum albumin antigen. In other embodiments, kits described herein contain one or more elements for detecting the binding of a recombinant protein to a serum albumin antigen (e.g., the recombinant protein can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized serum albumin antigen. The serum albumin antigen provided in the kit can also be attached to a solid support. In some embodiments, the detecting means of the above-described kits include a solid support to which a serum albumin antigen is attached. Such kits can also include a non-attached reporter-labeled anti-human antibody or anti-mouse/rat antibody. In binding of the antibody to the serum albumin, the antigen can be detected by binding of the said reporter-labeled antibody.

As described above, the recombinant fusion protein according to some aspects is expected to have an extended half-life in the form fused to SL335 (SAFA-IL-2m) compared to recombinant human IL-2m, and thus can exhibit similar efficacy with fewer number of dosing number, resulting in providing a more convenient drug dosing interval to patients.

Reference will now be made in detail to embodiments, examples of which are illustrated in the accompanying drawings, wherein like reference numerals refer to like elements throughout. In this regard, the present embodiments can have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, Examples are presented to help understanding of the present disclosure. However, Examples below are only presented for easier understanding of the present disclosure, and the contents of the present disclosure are not limited by the following examples.

### EXAMPLES

### Example 1. Preparation of Recombinant Fusion Protein Including Interleukin-2 Mutein and Antigen-Binding Fragment to Serum Albumin

### 1-1. Preparation of Expression Vectors for Interleukin-2 Mutein and Antigen-Binding Fragment to Serum Albumin

An expression vector for a recombinant fusion protein including interleukin-2 mutein and an antibody fragment binding to serum albumin was prepared. SL335H-linker-IL-2 ml genes (SEQ ID NO:3), in which interleukin-2 mutein 1 genes (C125A) (IL-2 m1, SEQ ID NO:1) and heavy chain genes of a Fab antibody fragment binding to serum albumin (SL335) (SEQ ID NO: 6) are linked by a peptide linker (SEQ ID NO:84), and SL335L-linker-IL-2m2 genes, in which interleukin-2 mutein 2 genes (N88R, V91D, M104S, C125A) (IL-2m2, SEQ ID NO:2) and heavy chain genes of a Fab antibody fragment binding to serum albumin (SL335) (SEQ ID NO: 6) are linked by a peptide linker (SEQ ID NO:84), were synthesized (SEQ ID NO:4) after undergoing gene codon optimization by Cosmogenetech Co., Ltd. (South Korea).

To express the SL335H-linker-IL-2 ml genes and the SL335L-linker-IL-2m2 genes produced by the gene synthesis, a pD2535NT (ATUM, USA) expression vector, the SL335H-linker-IL-2 ml genes, and the SL335L-linker-IL-2m2 genes were each treated with Bbs I restriction enzyme (Takara, Japan) to cleave a Bbs I site, and then were each treated with a T4 DNA ligase (Takara, Japan) and inserted into the expression vector.

The light chain genes of the Fab antibody fragment binding to serum albumin (SL335) were produced by the gene synthesis after undergoing gene codon optimization by ATUM Company (USA), and then inserted into the pD2359 expression vector (ATUM, USA).

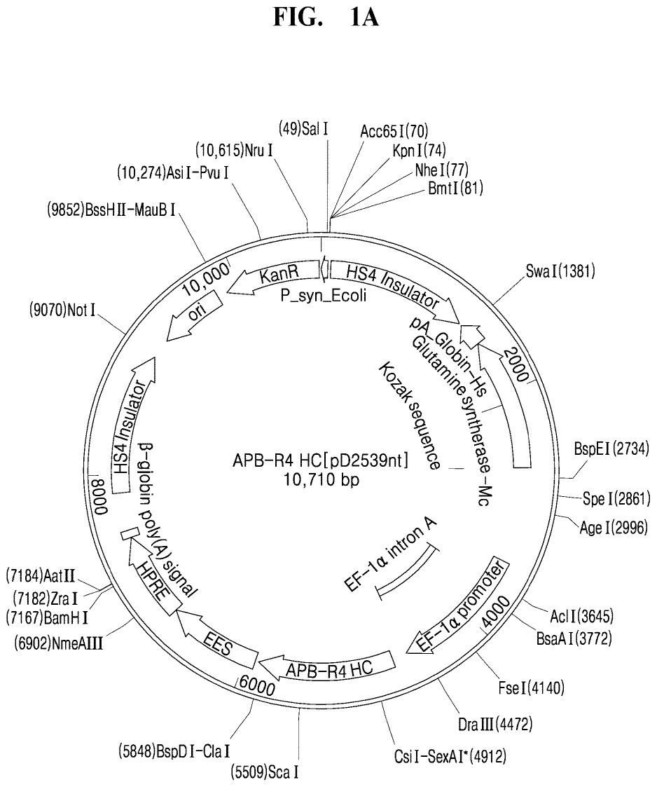

FIG. 1A shows an expression vector for a heavy chain and FIG. 1B shows an expression vector for a light chain to prepare a recombinant fusion protein according to some aspects.

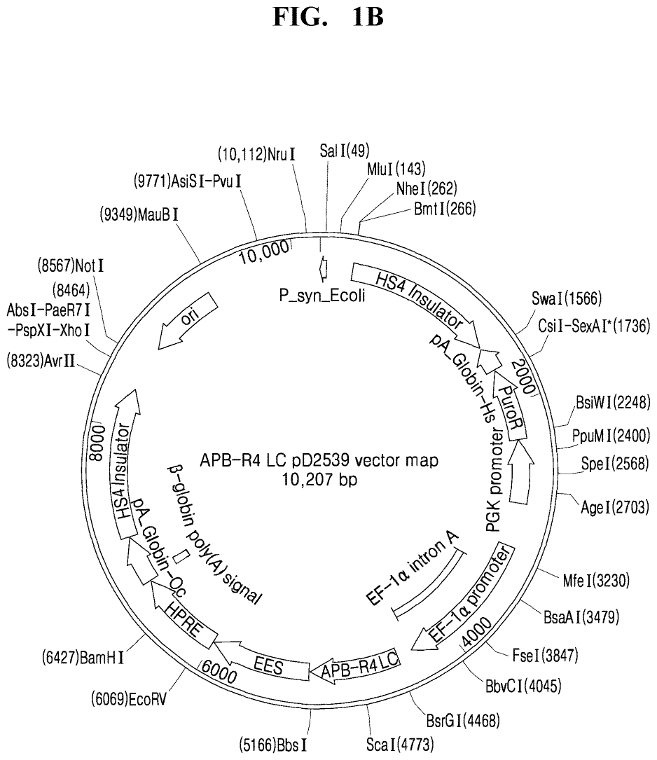

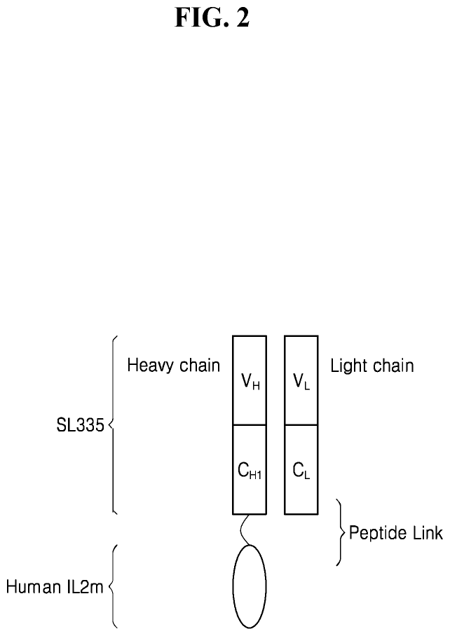

FIG. 2 shows the structure of an APB-R4 (SAFA-IL-2m2) protein to be prepared according to an aspect.

### 1-2. Preparation of Transient Expression Cells

After adding ExpiCHO-S™ cells (Thermo Fisher scientific) to a 125 ml culture flask containing an expression medium (ExpiCHO expression media, Thermo Fisher Scientific), the cells were cultured under conditions of a temperature of 37° C., 140 rpm, 5% CO₂, and 80% humidity. Afterwards, for the preparation of transient expression cells, the cultured cells were seeded at 6a concentration of 6.0×10⁶ cells/ml, and then transfected with plasmid vectors (pD2535NT and pD2539) to which each of the genes prepared in Example 1-1 were inserted. Then, transfected cells were cultured for 16 hours in a shaking incubator under conditions of a temperature of 37° C., 140 rpm, 5% CO₂, and 80% humidity, subsequently treated with ExpiCHO feed and an enhancer, and then, cultured under the same conditions as described above. On Day 6, D-glucose was additionally treated, and the resultant cells were cultured for 8 days under conditions of a temperature of 37° C., 140 rpm, 5% CO₂, and 80% humidity. After completion of the culture, the culture medium thus obtained was subjected to centrifugation under conditions of 4,000 rpm, 15 minutes, and 4° C., so as to separate the cells from the culture medium. Next, the separated culture medium was passed through a 0.2 μm filter paper to remove impurities.

### 1-3. Preparation of Stable Cell Lines

A stable cell line was prepared by using HD-BIOP3 GS null CHO-K1 cells (Horizon Discovery). Specifically, cells were seeded at a concentration of 3.0×10⁵ cells/ml into a CD FortiCHO (Thermo Fisher Scientific) medium supplemented with 4 mM of L-glutamine, and were subjected to seed culture in a shaking incubator under conditions of a temperature of 37° C., 5% CO₂, and humidity of 80% or higher. For transfection, the cultured cells were seeded at a concentration of 1.0×10⁶ cells/ml, and then transfected with the SAFA-IL-2 ml and SAFA-IL-2m2 plasmid vectors (pD2535NT and pD2539) prepared in Example 1-1 by using an OptiPRO SFM medium together with a Freestyle max reagent (Invitrogen, Carlsbad, California). Then, the transfected cells were cultured for 2 days under conditions of a temperature of 37° C., 5% CO₂, and humidity of 80% or higher. Afterwards, to proceed with stable pool selection, the medium was replaced with L-glutamine-free CD FortiCHO medium by centrifugation, and the cells were treated with 50 μM methionine sulfoximine (MSX) (Sigma-Aldrich, St. St. Louis, Missouri) and 10 μg/ml of puromycin (Thermo Fisher Scientific) every two days to remove cells that were not injected into the vector. Thereafter, by using a centrifuge, the medium was replaced with a CD FortiCHO medium containing both MSX and puromycin at intervals of 7 to 10 days, and the cells were cultured for 21 days while maintaining the number of cells at 5.0×10⁵ cells/ml each time. Afterwards, when the viability was recovered to 90% or more, a stock of 1.0×10⁷ cells/ml was prepared.

### 1-4. Isolation and Purification of SAFA-IL-2 Mutein

Protein samples present in the SAFA-IL-2 ml and SAFA-IL-2m2 stable cell lines of Example 1-3 were purified by sequentially performing affinity chromatography (AC), multimodal anion exchange chromatography (MAEX), and cation exchange chromatography (CEX).

Specifically, to purify the SAFA-IL-2 ml and SAFA-IL-2m2 (APB-R4) proteins of Example 1-3 by AC, the culture medium was equilibrated with 1×PBS (pH 7.4) by using a CaptureSelect CH1-XL resin (Thermo fisher science, USA), and then, was combined at a fluid speed of 136 cm/hr, thereby eluting the proteins with a 25 mM sodium citrate pH 5.0 buffer. The eluted protein solution was subjected to MAEX using a Capto adhere ImpRes resin (Cytiva, USA) to remove impurities by FT mode at pH 6.0. The separated protein solution was finally combined with a resin equilibrated with a 20 mM L-histidine buffer pH 7.0 by using CEX with Capto SP ImpRes resin (Cytiva, USA), and the final protein was separated and purified by using a 20 mM L-Histidine buffer pH 7.0, 1M NaCl. The proteins thus purified were named SAFA-IL-2 ml and SAFA-IL-2m2 (APB-R4).

### Example 2. Preparation of Recombinant Human IL-2 Mutein

### 2-1. Preparation of Expression Vector for IL-2 Mutein, Efavaleukin-Alfa Analogue

An expression vector expressing an efavaleukin-alfa (AMGEN, AMG-592) analogue protein, which is a comparative substance of IL-2 mutein, was prepared. The efavaleukin-alfa analogue was synthesized (SEQ ID NO:9) after gene codon optimization by Cosmogenetech Co., Ltd. (South Korea) using the public sequence information from the drug information site (KEGG, Kyoto Encyclopedia of Genes and Genomes, D11612).

To express the efavaleukin-alfa analogue genes produced by the gene synthesis, a pD2535NT expression vector (ATUM, USA) and the efavaleukin-alfa analogue gene were treated with a BbsI restriction enzyme (Takara, Japan) to cleave a BbsI site, and then were each treated with a T4 DNA ligase (Takara, Japan) and inserted into the expression vector.

### 2-2. Preparation of Transient Expression Cells

After adding ExpiCHO-S™ cells (ThermoFhisher scientific) to a 125 ml culture flask containing an expression medium (ExpiCHO expression media, Thermo Fisher Scientific), the cells were cultured under conditions of a temperature of 37° C., 140 rpm, 5% CO₂, and 80% humidity. Afterwards, for the preparation of transient expression cells, the cultured cells were seeded at 6a concentration of 6.0×10⁶ cells/ml, and then transfected with a plasmid vector (pD2535NT) to which the genes prepared in Example 2-1 were inserted. Then, transfected cells were cultured for 16 hours in a shaking incubator under conditions of a temperature of 37° C., 140 rpm, 5% CO₂, and 80% humidity, subsequently treated with ExpiCHO feed and an enhancer, and then, cultured under the same conditions as described above. On Day 6, D-glucose was additionally treated, and the resultant cells were cultured for 8 days under conditions of a temperature of 37° C., 140 rpm, 5% CO₂, and 80% humidity. After completion of the culture, the culture medium thus obtained was subjected to centrifugation under conditions of 4,000 rpm, 15 minutes, and 4° C., so as to separate the cells from the culture medium. Next, the separated culture medium was passed through a 0.2 μm filter paper to remove impurities.

### 2-3. Preparation of Stable Cell Lines

A stable cell line was prepared by using HD-BIOP3 GS null CHO-K1 cells (Horizon Discovery). Specifically, cells were seeded at a concentration of 3.0×10⁵ cells/ml into a CD FortiCHO (Thermo Fisher Scientific) medium supplemented with 4 mM of L-glutamine and were subjected to seed culture in a shaking incubator under conditions of a temperature of 37° C., 5% CO₂, and humidity of 80% or higher. For transfection, the cultured cells were seeded at a concentration of 1.0×10⁶ cells/ml, and then transfected with the efavaleukin-alfa analogue plasmid vector (pD2535NT) prepared in Example 2-1 by using an OptiPRO SFM medium together with a Freestyle max reagent (Invitrogen, Carlsbad, California). Then, the transfected cells were cultured for 2 days under conditions of a temperature of 37° C., 5% CO₂, and humidity of 80% or higher. Afterwards, to proceed with stable pool selection, the medium was replaced with L-glutamine-free CD FortiCHO medium by centrifugation, and the cells were treated with 50 μM methionine sulfoximine (MSX) (Sigma-Aldrich, St. St. Louis, Missouri) and 10 μg/ml of puromycin (Thermo Fisher Scientific) every two days to remove cells that were not injected into the vector. Thereafter, by using a centrifuge, the medium was replaced with a CD FortiCHO medium containing both MSX and puromycin at intervals of 7 to 10 days, and the cells were cultured for 21 days while maintaining the number of cells at 5.0×10⁵ cells/ml each time. Afterwards, when the viability was recovered to 90% or more, a stock of 1.0×10⁷ cells/ml was prepared.

### 2-4. Isolation and Purification of Efavaleukin-Alfa Analogue Protein

To purify the efavaleukin-alfa analogue protein of Example 2-3 by AC, the culture medium was equilibrated with 1×PBS (pH 7.4) by using a CaptureSelect CH1-XL resin (Thermo fisher science, USA), and then, was combined at a fluid speed of 240 cm/hr, thereby eluting the proteins with a 50 mM sodium citrate pH 3.0 buffer. The eluted protein solution was subjected to AEX using a POROS™ XQ resin (Thermo fisher science, USA) to remove impurities by FT mode at pH 5.5. The separated protein solution was finally combined with a resin equilibrated with a 25 mM Sodium citrate pH 5.0 by using CEX with CM Sepharose FF resin (Cytiva, USA), and the final protein was separated and purified by using a 5 mM sodium citrate pH 5.0, 1M NaCl.

### Example 3. Molecular Characterization of SAFA-IL-2m

### 3-1. Size Confirmation

To confirm the size of the recombinant proteins of Example 1 and Example 2, sodium dodecyl sulfate polyacrylamide gel electrophoresis (SDS-PAGE) was performed thereon. Specifically, protein samples were prepared under reducing conditions and non-reducing conditions by using a non-reducing 4×SDS sample buffer (Thermo Fisher Scientific) and 2-mercaptoethanol. In the case of non-reducing conditions, samples heated at 100° C. for 5 minutes and samples not heated were prepared together to compare the shape and size of proteins depending on heat. To compare the size of proteins in each condition, a protein size marker (SMOBio, Taiwan) was prepared. The prepared protein samples were each loaded on a 4-15% Mini-protein TGX precast gel (15-well, by Bio-Rad) to be contained in 1 μg or 2 μg per well, and then subjected to electrophoresis in a tris-glycine SDS running buffer at 150 voltage (V) for 1 hour. After completion of the electrophoresis, the SDS-PAGE gel was stained with EZ-gel staining solution (DoGenBio, South Korea) for 1 hour, followed by destaining in distilled water for 1 day.

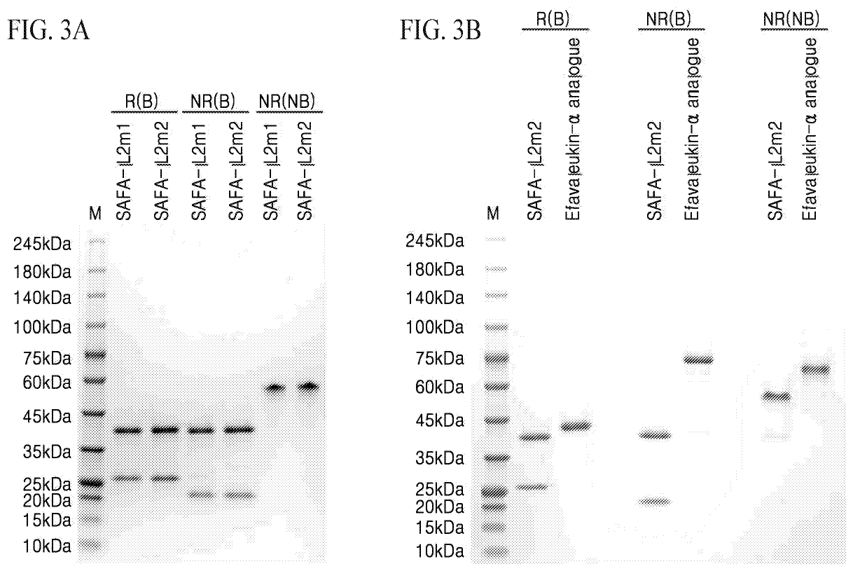

FIG. 3A shows a result of analyzing the size of the SAFA-IL-2 ml and SAFA-IL-2m2 proteins according to an aspect, in 1 μg per well by SDS-PAGE under the reducing (R), non-reducing (NR(B)) with heat, and non-reducing (NR(NB)) without heat conditions;

FIG. 3B shows a result of analyzing the size of the SAFA-IL-2m2 and efavaleukin-alfa analogue proteins according to an aspect, in 1 μg per well by SDS-PAGE under the reducing (R), non-reducing (NR(B)) with heat, and non-reducing (NR(NB)) without heat conditions.

As a result, as shown in FIG. 3A, in the reducing condition and the non-reducing condition with heat applied for 5 minutes of the SAFA-IL-2 ml and SAFA-IL-2m2 proteins, the protein bands were observed at 40 kDa, which is similar to the theoretical size of 39.6 kDa, in the heavy chain of the SL335H-IL-2 ml and SL335H-IL-2m2, and the protein bands were observed at 26.3 kDa, which is similar to the theoretical size of 23.3 kDa, in the light chain of the SL335L. In the non-reducing condition, changes in protein size caused by O-linked glycans present in the IL-2m protein were not confirmed. Meanwhile, in the non-reducing conditions with heat not applied, the protein bands corresponding to the intact form of APB-R4 in which the heavy and light chains were bound were observed with high purity at 56.7 kDa, which is smaller to the theoretical size of 62.975 kDa, whereas the protein bands corresponding to each of the heavy chain and the light chain were detected very weakly.

In the case of the efavaleukin-alfa analogue, as shown in FIG. 3B, the protein bands were observed at 43.3 kDa, which is similar to the theoretical size of 41.1 kDa, in the reducing condition and the non-reducing condition with heat applied for 5 minutes, whereas the protein bands corresponding to the intact form of efavaleukin-alfa analogue in which the heavy and light chains were bound were observed with high purity at 69.8 kDa, which is lower than the theoretical size of 82.196 kDa, in the non-reducing condition without heat applied. Also, the protein bands corresponding to efavaleukin-alfa analogue monomer in which the heavy and light chains were not bound were detected very weakly.

### 3-2. Purity Confirmation

To measure purity of the SAFA-IL-2m2 (APB-R4) and efavaleukin-alfa analogue proteins purified in Example 1-4 and Example 2-4, SE-HPLC was performed thereon. First, HPLC equipment with a TSKgel G3000SWXL 7.8×300 mm (Tosoh Bioscience, Japan) column and an Alliance HPLC system (Waters, Milford, MA) was subjected to equilibration with a 300 mM sodium phosphate pH 7.0 buffer containing 100 mM KCl. A sample to be analyzed was prepared by dilution with 300 mM potassium phosphate pH 7.0, 100 mM KCl, and a pH 7.0 buffer, and 50 μg of the prepared sample was loaded onto the column. The SE-HPLC analysis was performed for 40 minutes under conditions of a flow rate of 0.5 ml/min and a maximum pressure of 1,000 psi, and the purity was measured at a wavelength of A280 nm.

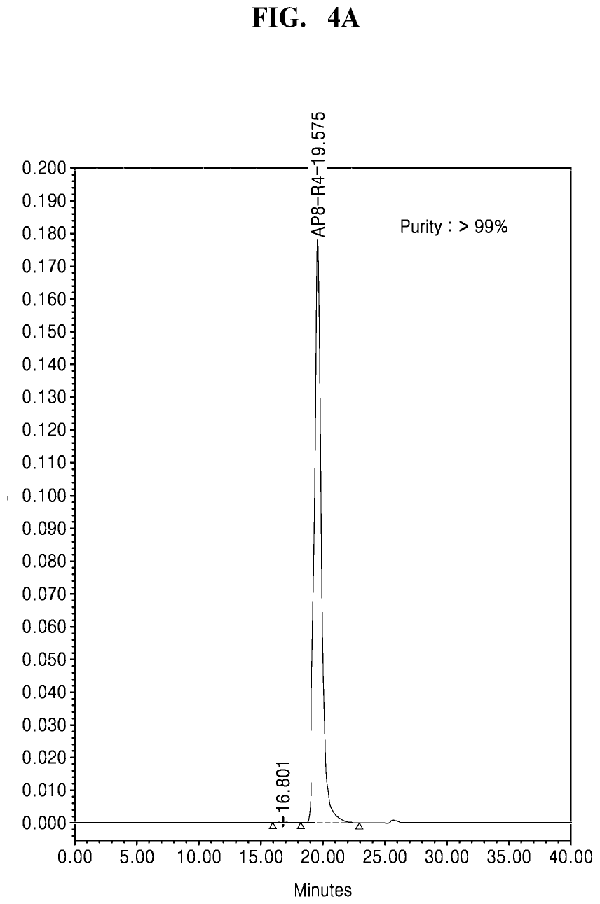

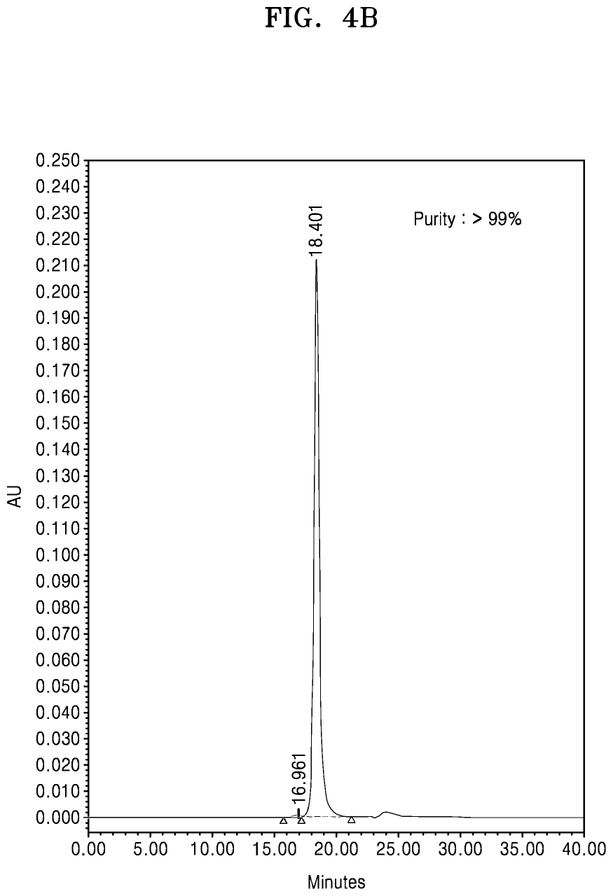

FIGS. 4A and 4B show the results of analyzing the purity of the SAFA-IL-2m2 and efavaleukin-alfa analogue proteins according to an aspect, by SE-HPLC. The SAFA-IL-2m2 and efavaleukin-alfa analogue proteins were all confirmed to have purity of 99% or more.

### Example 4. Confirmation of Biological Properties of SAFA-IL-2m

### 4-1. Analysis of Binding Ability of SAFA-IL-2m to Human Serum Albumin, Monkey Serum Albumin, and Mouse Serum Albumin

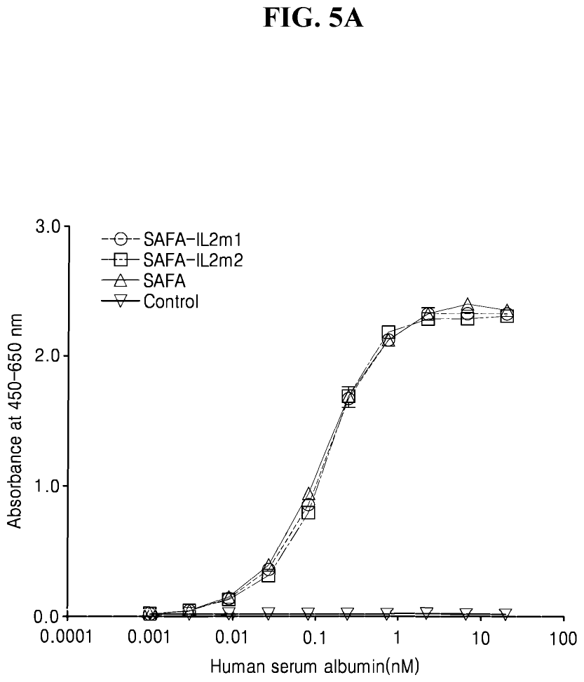

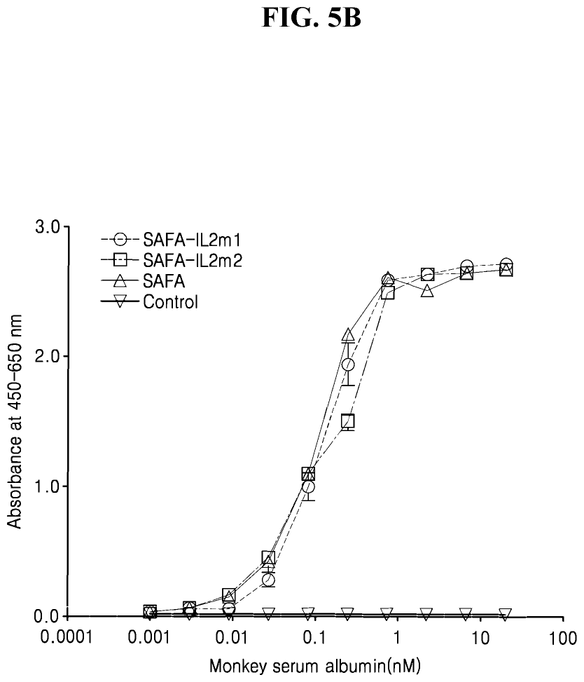

To evaluate the biological function of the SAFA-IL-2m2 protein purified in Example 1-4, the binding ability of the SAFA-IL-2m2 protein to human serum albumin, monkey serum albumin, and mouse serum albumin was confirmed by ELISA method. As shown in FIGS. 5A-5C, it was confirmed that the SAFA-IL-2m2 does not interfere with the cross-reactivity of SAFA to serum albumin.

### 4-2. Confirmation of Binding Degree of IL-2 Receptor at Cellular Level

To evaluate the biological function of the SAFA-IL-2m2 protein purified in Example 1-4, the HEK-blue IL-2 reporter cell line expressing all the IL-2 receptors alpha, beta, and gamma, and the HEK-blue IL-2 reporter cell line expressing both IL-2 receptors beta and gamma were used to confirm the IL-2 receptor-dependent activity of the SAFA-IL-2m2 protein. First, the protein sample of Example 1 was prepared by diluting in a PBS buffer mixed with 0.2% bovine serum albumin (BSA). Then, cells were prepared at a concentration of 2.777×10⁵ cells/ml, and the diluted protein sample was seeded thereon. The mixed cells and protein reacted for 21 hours in an incubator under conditions of a temperature of 37° C. and 5% CO₂. After the reaction, the supernatant of the cells mixed with the protein was mixed with a QUANTI-blue solution, and the resultant mixture was reacted for 1 hour in an incubator under the conditions of a temperature of 37° C. and 5% CO₂. Then, the absorbance was measured at a wavelength of 655 nm.

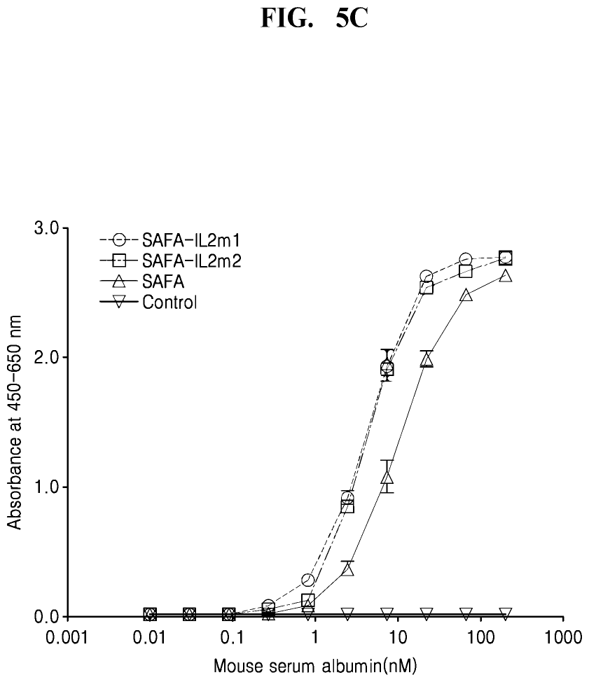

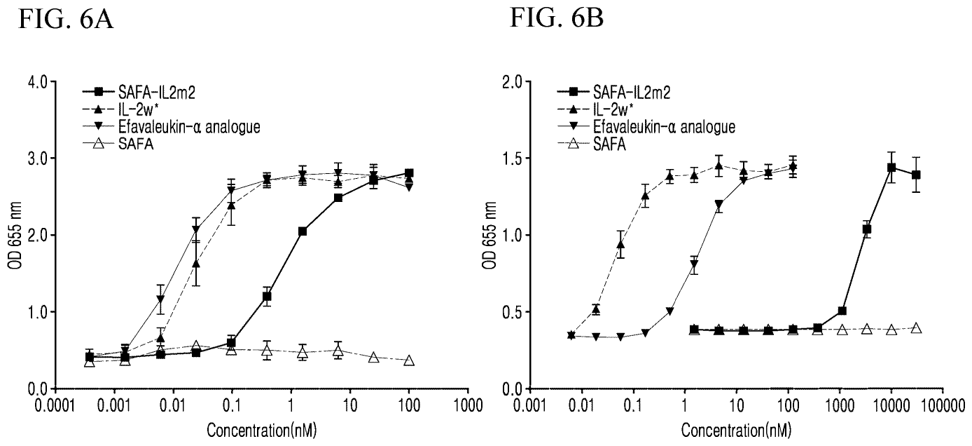

As a result, the IL-2 mutant was demonstrated based on the results that the activity of the SAFA-IL-2m2 was lower than that of the efavaleukin-alfa analogue in the cell line expressing all of the IL-2 receptors alpha, beta, and gamma as shown in FIG. 6A and the results that the SAFA-IL-2m2 did not show activity in the cell line expressing the IL-2 receptors beta and gamma as shown in FIG. 6B.

### 4-3. Confirmation of Binding Degree of SAFA-IL-2m2 to IL-2 Receptor

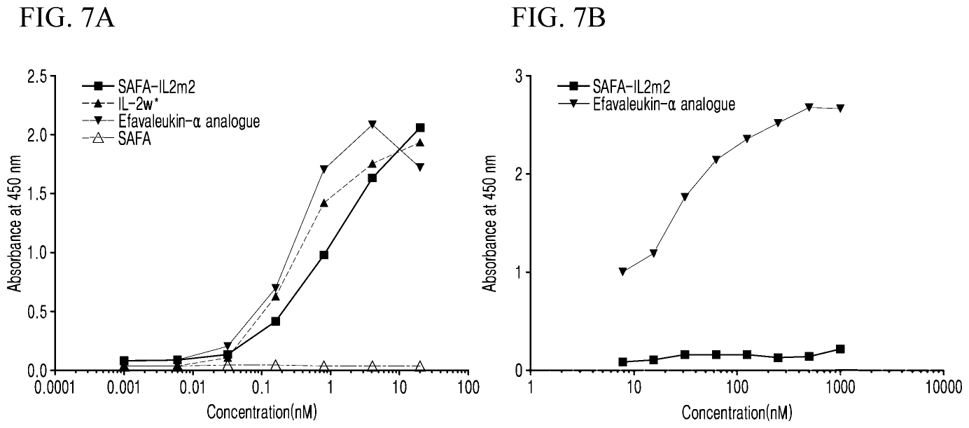

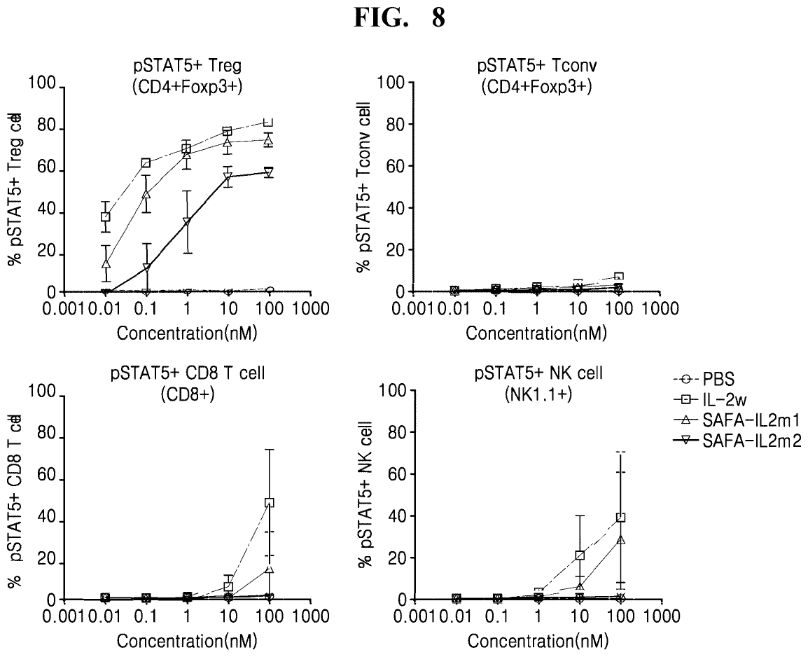

In FIGS. 7A and 7B, the binding ability of the SAFA-IL-2m2 protein to the IL-2 receptors alpha and beta was confirmed by ELISA analysis method.

As a result, it was confirmed that the SAFA-IL-2m2, which has less mutations for the IL-2 receptor alpha, had reduced binding ability compared to the IL-2 wild-type and the efavaleukin-alfa analogue, and that the SAFA-IL-2m2, which has large mutations for the IL-2 receptor beta, had no binding compared to the IL-2 wild type and the efavaleukin-alfa analogue.

### 4-4. Verification of Specific Activity of Regulatory T Cells

The SAFA-IL-2m2 protein purified in Example 1-4 was aimed to impart specificity that completely reduces the signaling ability of IL-2 through the intermediate-affinity-type IL-2 receptor complex of beta and gamma and that reduces the signaling ability of IL-2 through the IL-2 receptor complex of alpha, beta, and gamma with affinity. In this regard, the ability of the SAFA-IL-2m2 protein to differentially induce signal transducer and activation of transcription 5 (STAT5) phosphorylation in regulatory T cells, CD4+ Tconv cells, CD8+ T cells, and NK cells was investigated. The STAT5 is known to be involved in the downstream signaling cascade when IL-2 binds to the IL-2 receptors.

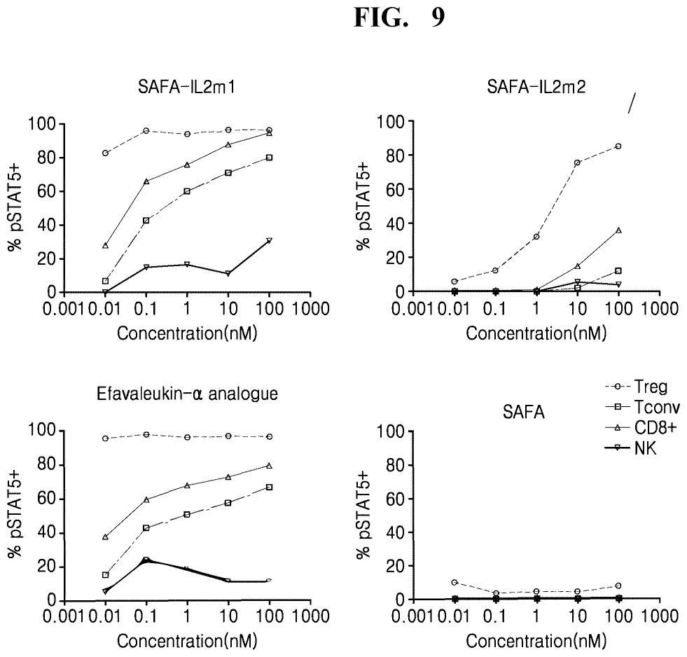

Briefly, cells separated from the spleen of C57BL/6 mice (available from Orient Bio Co., Ltd.) were treated with the SAFA-IL-2m2 for 15 minutes at different concentration, fixed and permeabilized, washed, and then, stained with a mixture of anti-CD45-BV510, anti-CD3−BV605, anti-CD4−PerCP/cy5.5, anti-CD8−BV786, anti-CD25−PE, anti-FOXP3-AF700, anti-NK1.1-AF488, and anti-PSTAT5-AF647 antibodies. Then, by flow cytometric analysis, the cells were gated into CD4+CD25+Foxp3+, CDF4+CD25+Foxp3−, CD3+CD8+, CD4−CD8− groups for regulatory T cells, CD4+ Tconv cells, CD8+ T cells, and NK cells, respectively. Data are expressed as percentage of the pSTAT5 positive cells in the gated population, and are illustrated in FIG. 8. It was verified that the phosphorylation of STAT5 was increased specifically by concentrations of the SAFA-IL-2m2 protein only in regulatory T cells.

As shown in FIG. 9, the phosphorylation of STAT5 using human peripheral blood mononuclear cells (PBMCs) was confirmed by a flow cytometer in the same T cell population as shown in FIG. 8.

Briefly, 3×10⁶ human PBMCs were reacted for 2 hours in an incubator under conditions of a temperature of 37° C. and 5% CO₂, treated with 100 ng/mL anti-CD3 antibody, and then allowed for a reaction for two days in an incubator under conditions of a temperature of 37° C. and 5% CO₂. Next, the human PBMCs were prepared under conditions of a concentration of 2×10⁶, and allowed for a reaction for 5 days in an incubator under conditions of a temperature of 37° C. and 5% CO₂. Next, 0.5×10⁶ human PBMCs were treated with serial dilutions of a test compound for 15 minutes in an incubator under conditions of a temperature of 37° C. and 5% CO₂. Cells were fixed and permeabilized, washed, and stained with a mixture of anti-CD45-PE/Cy7, anti-CD3-BV605, anti-CD4-FITC, anti-CD8-PE, anti-CD25-BV421, anti-FOXP3-AF700, anti-CD49b-CD510, and anti-pSTAT5-AF547 antibodies. Then, by flow cytometric analysis, the cells were gated into CD4+CD25+Foxp3+, CDF4+CD25+Foxp3−, CD3+CD8+, CD4−CD8− groups for regulatory T cells, CD4+ Tconv cells, CD8+ T cells, and NK cells, respectively. Data are expressed as percentage of the pSTAT5 positive cells in the gated population, and are illustrated in FIG. 9. It was verified that the efavaleukin-alfa analogue, which is a control substance, caused phosphorylation of STAT5 not only in regulatory T cells, but also in CD4+ Tconv cells, CD8+ T cells, and NK cells, whereas the SAFA-IL-2m2 protein caused phosphorylation of STAT5 only in regulatory T cells.

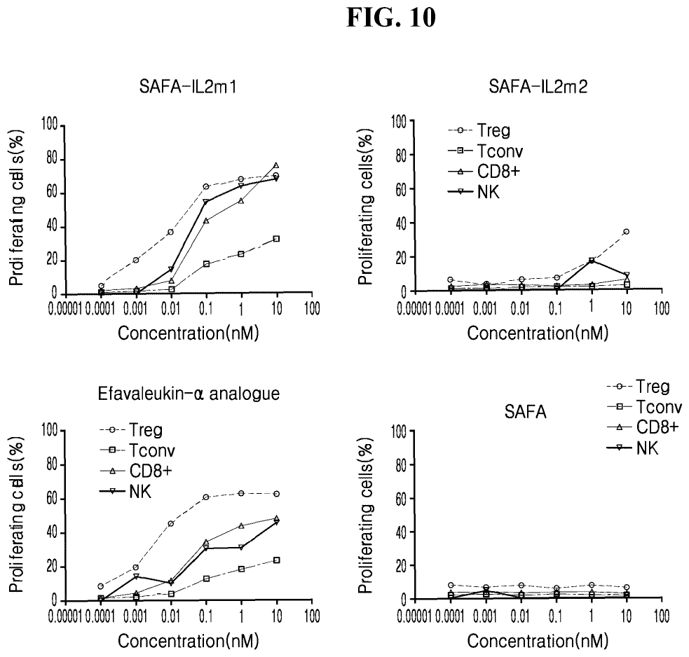

As shown in FIG. 10, the changes in Ki67 proliferation marker using human PBMCs were confirmed by a flow cytometer in the same T cell population as shown in FIG. 8.

Briefly, 3×10⁶ human PBMCs were reacted for 2 hours in an incubator under conditions of a temperature of 37° C. and 5% CO₂, treated with 100 ng/mL anti-CD3 antibody, and then allowed for a reaction for two days in an incubator under conditions of a temperature of 37° C. and 5% CO₂. Next, the human PBMCs were prepared under conditions of a concentration of 2×10⁶, and allowed for a reaction for 5 days in an incubator under conditions of a temperature of 37° C. and 5% CO₂. Next, 0.5×10⁶ human PBMCs were treated with serial dilutions of a test compound for two days in an incubator under conditions of a temperature of 37° C. and 5% CO₂. Cells were fixed and permeabilized, washed, and stained with a mixture of anti-CD45-PE/Cy7, anti-CD3-BV510, anti-CD4-FITC, anti-CD8-PerCP/Cy5.5, anti-CD25−APC, anti-FOXP3-eFluor450, anti-CD49b-BV510 and anti-ki67-PE antibodies. Then, by flow cytometric analysis, the cells were gated into CD4+CD25+Foxp3+, CDF4+CD25+Foxp3−, CD3+CD8+, CD4-CD8− groups for regulatory T cells, CD4+ Tconv cells, CD8+ T cells, and NK cells, respectively. Data are expressed as percentage of the Ki67 positive cells in the gated population, and are illustrated in FIG. 10. It was observed that the efavaleukin-alfa analogue, which is a control substance, stimulated the proliferation and spread not only in regulatory T cells, but also in CD4+ Tconv cells, CD8+ T cells, and NK cells, whereas the SAFA-IL-2m2 protein strongly caused proliferation and spread only in regulatory T cells.

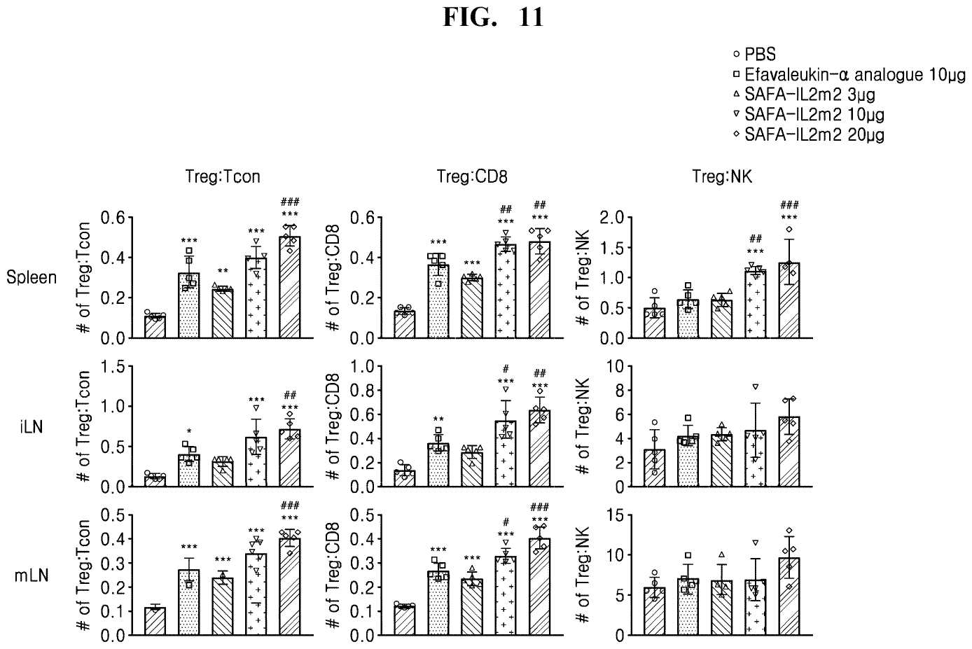

As shown in FIG. 11, the changes in the proliferation of regulatory T cells were confirmed by a flow cytometer by at the time of a single subcutaneous injection to the C57BL/6 mice, after separating cells from the spleen, inguinal lymph node, and mesenteric lymph node of the mice.

As a result, as shown in FIG. 11, it was confirmed that the ratio of the number of regulatory T cells to the number of CD4+ Tconv cells, the ratio of the number of regulatory T cells to the number of CD8+ T cells, and the ratio of the number of regulatory T cells to the number of NK cells increased in a SAFA-IL-2m2 concentration dependent manner. Also, it was confirmed that the formation of regulatory T cells was superior in the SAFA-IL-2m2 to the efavaleukin-alfa analogue.

### 4-5. Validation of Efficacy of SAFA-IL-2m2

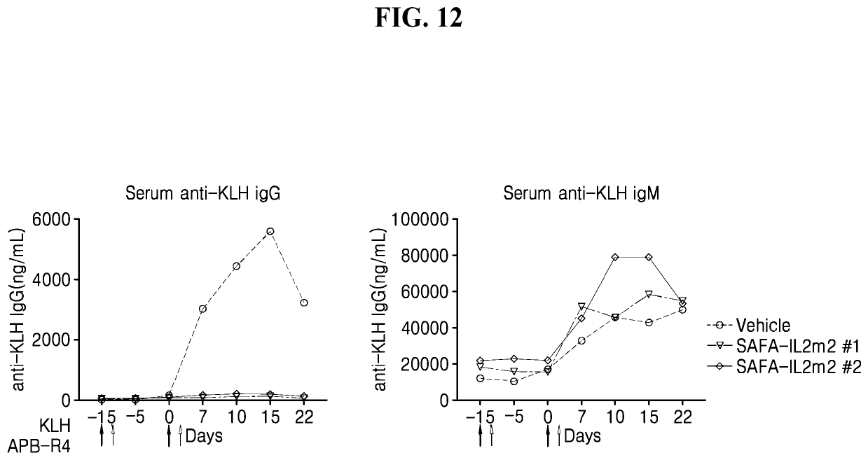

FIG. 12 shows a result of validating the immune response through administration of the SAFA-IL-2m2 to a marmoset monkey, which is a type of primates, after immunization with keyhole limpet hemocyanin (KLH). The immune response was confirmed by ELISA on anti-KLH IgG and anti-KLH-IgM.

As a result, as shown in FIG. 12, it was confirmed that the levels of anti-KLH-IgG and anti-KLH-IgM were increased by the KLH immune response in the group administered only with KLH, and that no change was observed in the group administered with the SAFA-IL-2m2.

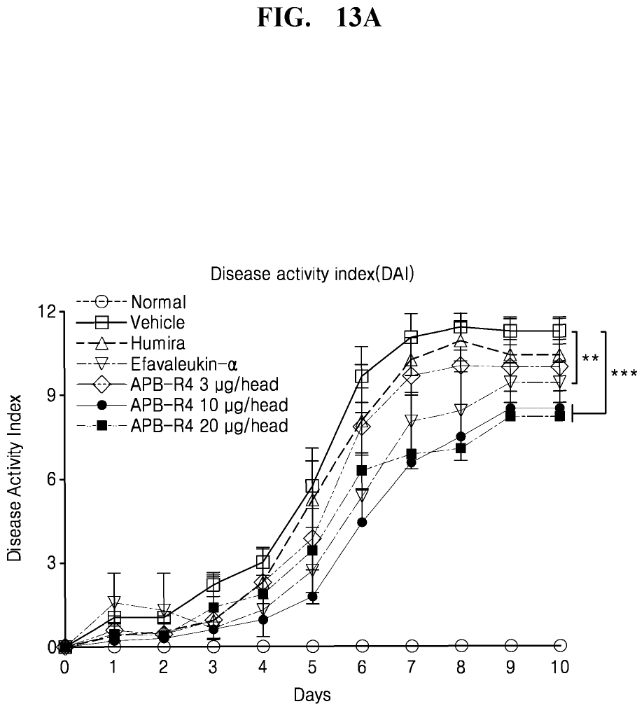

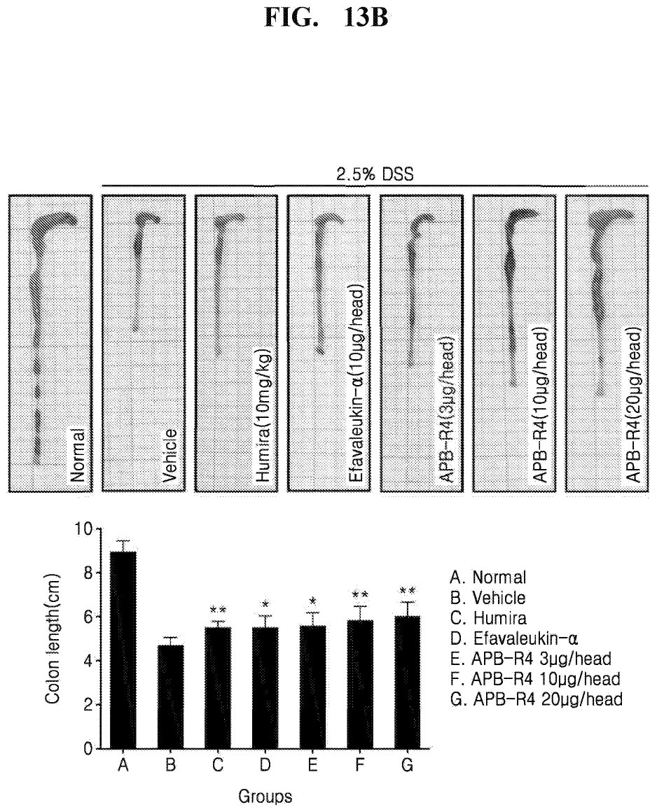

In FIGS. 13A and 13B, the effectiveness of the SAFA-IL-2m2 in the mouse model having inflammatory bowel disease induced by DSS was confirmed.

Briefly, as shown in FIG. 13A, the disease activity index (DAI) was quantitatively evaluated based on the occurrence of intestinal symptoms and weight change after the start of administration of the control substance (Humira, efavaleukin-alfa analogue) and the SAFA-IL-2m2. As a result of the evaluation, it was confirmed that, when the SAFA-IL-2m2 was administered at different concentrations, the DAI was tended to be statistically significantly reduced compared to the negative control group (vehicle), and that the DIA was tended to be low even compared to the positive control group (Humira, efavaleukin-alfa analogue).

As shown in FIG. 13B, the results obtained by measuring the length of the mouse colon tissue extracted at the end of the experiment. The length of the colon was decreased in the negative control group compared to the normal group, and the length of the colon was maintained at each concentration in the SAFA-IL-2m2-administered group. In this regard, this is interpreted in a way that the entire length of the colon was less decreased by the treatment.

### Example 5. Pharmacokinetic Evaluation of SAFA-IL-2m2 Protein

To measure the in vivo half-life of the SAFA-IL-2m2, pharmacokinetic experiments were performed on a cynomolgus monkey model and a mouse model (C57BL/6). Specifically, 12 healthy male monkeys (cynomolgus monkeys) were divided into 4 groups of 3 monkeys per group. The SAFA-IL-2m2 was administered at a single intravenous dose of 0.1 mg/kg (Group 1), a single intravenous dose of 0.3 mg/kg (Group 2), a single subcutaneous dose of 0.1 mg/kg (Group 3), or a single subcutaneous dose of 0.3 mg/kg (Group 4). Afterwards, 1 mL of whole blood was collected according to the set blood collection schedule [e.g., single intravenous administration at 0, 0.25, 0.5, 1, 8, 24, 48, 96, 168, 240, 360, 528, 696, and 864 hours (total of 14 points); and single subcutaneous administration at 0, 1, 8, 12, 24, 48, 72, 96, 120, 168, 240, 360, 528, 696, and 864 hours (total of 15 points)], and serum was separated by centrifugation and stored in a cryogenic freezer (−70° C.). Next, ELISA was performed to measure the concentration of the SAFA-IL-2m2 present in serum. For pharmacokinetic experiments using a mouse model, 30 female C57BL/6 mice were intravenously injected once at a dose of 0.4 mg/kg of the SAFA-IL-2m2. After the injection, blood samples were collected at 10 points in total, including 1 point before the administration and 9 points at 0.25, 0.5, 2.5, 5, and 10 hours, respectively on 1, 3, 5, and 7 days after the administration. The concentration of the SAFA-IL-2m2 present in the mouse serum was measured by ELISA.

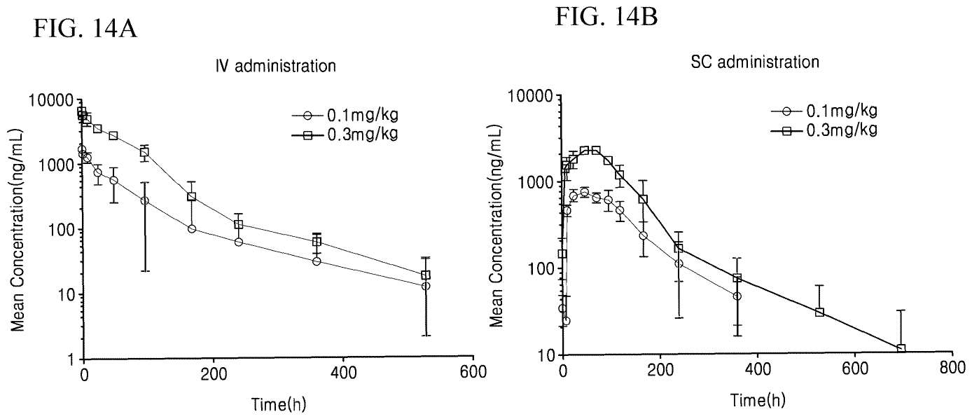

Next, after the intravenous and subcutaneous administration of the SAFA-IL-2m2 to the monkeys, pharmacokinetic parameters were determined by using a Phoenix WinNonlin software (ver 1.4; Certara L P, Princeton, NJ, USA) based on the result of measuring the protein concentration in blood. As shown in FIGS. 14A and 14B, AUCₗₐₛₜ was 88,000 hr*ng/ml, Cₘₐₓ was 1,670 ng/mL, and t_{1/2} was 64.6 hr in the group administered intravenously at a dose of 0.1 mg/kg. AUCₗₐₛₜ was 397,000 hr*ng/mL, Cₘₐₓ was 6,730 ng/mL, and t_{1/2} was 118 hr in the group administered intravenously at a dose of 0.3 mg/kg. AUCₗₐₛₜ was 113,000 hr*ng/mL, Cₘₐₓ was 772 ng/mL, and t_{1/2} was 71.5 hr in the group administered subcutaneously at a dose of 0.1 mg/kg, and AUCₗₐₛₜ was 321,000 hr*ng/mL, Cₘₐₓ was 2,370 ng/mL, and t_{1/2} was 114 hr in the group administered subcutaneously at a dose of 0.3 mg/kg.

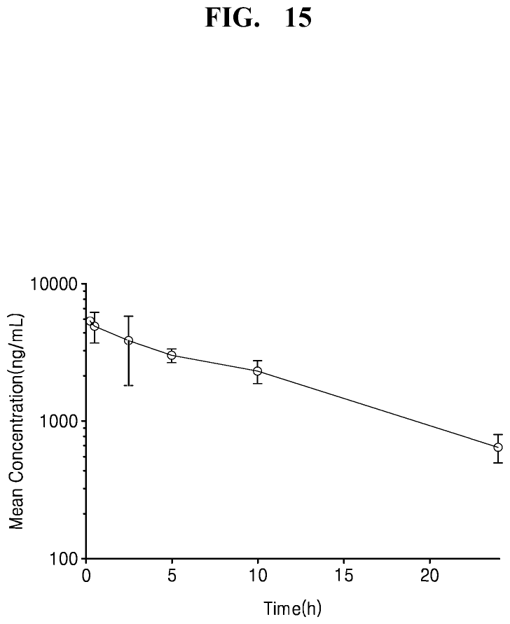

Also, after the single intravenous administration of the SAFA-IL-2m2 to mice, pharmacokinetic parameters were determined by using a Phoenix WinNonlin software (ver 8.3; Certara L P, Princeton, NJ, USA) based on the result of measuring the protein concentration in blood. As a result, as shown in FIG. 15, the SAFA-IL-2m2 had AUCₗₐₛₜ of 54,098 hr*μg/mL, C₀ of 5,827 μg/mL, and t_{1/2} of 8.3 hr.

### Example 6. Verification of Specific Activity to Regulatory T Cells

As verified for the specific activity of the SAFA-IL-2m2 protein on regulatory T cells in the C57BL/6 mouse model, experiments using three healthy male monkeys (cynomolgus monkeys) were carried out to verify the same effect in the monkey model. Specifically, the SAFA-IL-2m2 was administered subcutaneously once at a dose of 0.3 mg/kg. Next, 2 mL of whole blood was collected according to a predetermined blood collection schedule (at 0, 48, 96, 168, and 360 hours), and changes in the proliferation of regulatory T cells were confirmed by using a flow cytometer. By flow cytometric analysis, the cells were gated into CD45+CD3+, CD45+CD3+CD4+CD25+FoxP3+, CD45+CD3+CD4+CD25-FoxP3−, CD45+CD3+CD4−CD8+CD45+CD3−CD16+ groups for total T cells, regulatory T cells, helper T cells, killer T cells, and natural killer T cells, respectively.

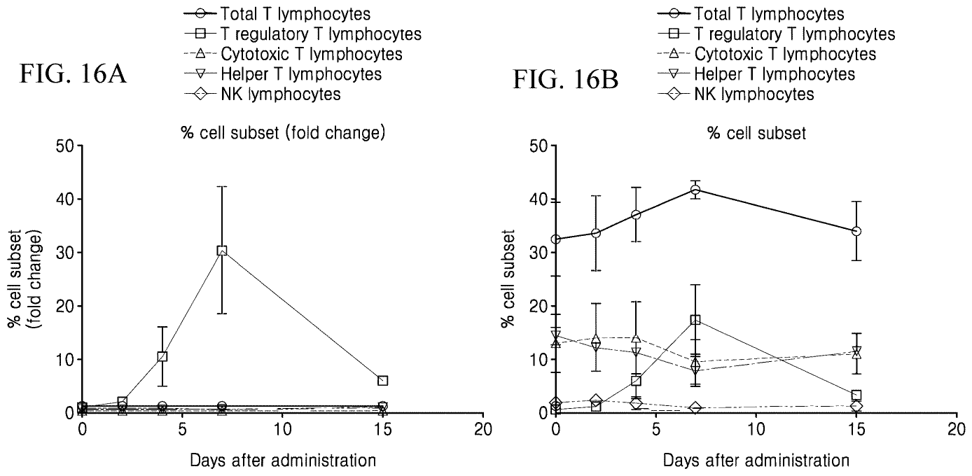

As shown in FIGS. 16A and 16B, it was observed that proliferation was strongly increased in only regulatory T cells by the SAFA-IL-2m2 administration, whereas and proliferation of other T cells was not increased.

### Example 7. Analysis of Bio-Distribution of SAFA-IL-2m2 Protein

For the purpose of evaluating the bio-distribution of the SAFA-IL-2m2 in inflammatory arthritis by a DBA/1J mouse model having collagen-induced arthritis, the SAFA-IL-2m2 was administered subcutaneously once to the mouse model having collagen-induced arthritis, and then, the bio-distribution of the SAFA-IL-2m2 over time was verified through imaging analysis. A second immunization was carried on Day 21 after a first immunization to DBA/1J male mice (8 to 9 weeks-old), and six animals per group were assigned as experimental groups. Periodically from the date of the group separation to the end of the experiment, the occurrence of inflammation and clinical arthritis index (CAI) thereof were observed to perform clinical evaluation of arthritis activity index. At the time of highest CAI, subcutaneous injection was made with 3 mg/kg of vehicle-CF750 (Group 1), 6.5 mg/kg of efavaleukin-alfa-CF750 (Group 2), and 5 mg/kg of SAFA-IL-2m2 (Group 3). After being anesthetized with inhalational anesthetic, optical images were taken with IVIS Lumina (PerkinElmer) at 0, 1, 4, 6, 24, 48, and 72 hours. After 72 hours of the administration of a test substance, the liver, lung, spleen, and kidney were removed from the mouse and analyzed for bio-distribution.

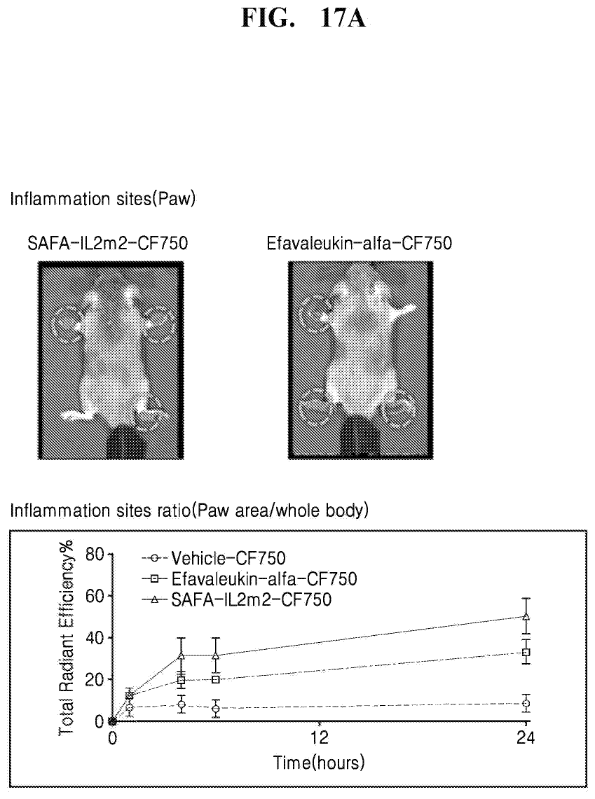

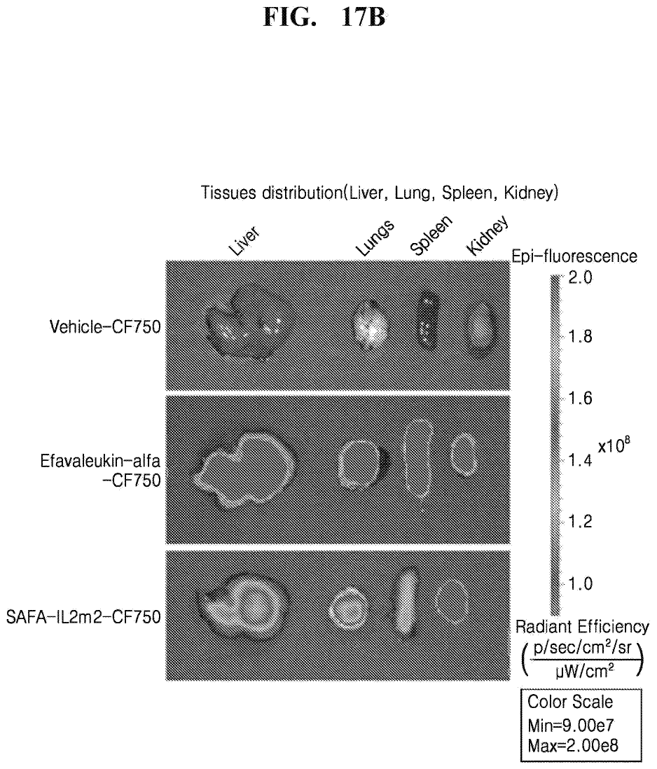

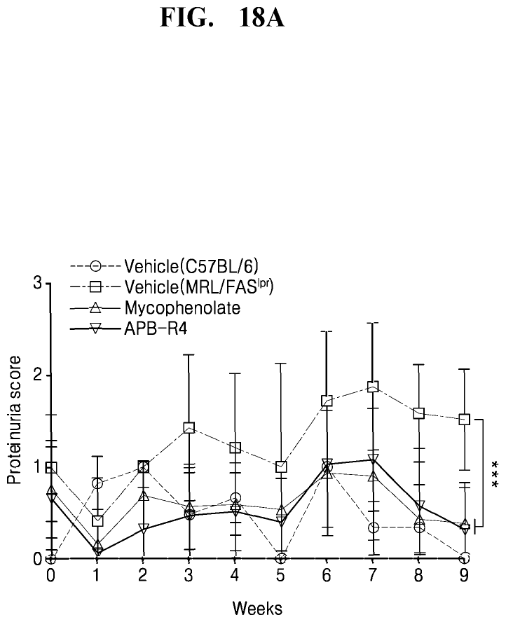

As shown in FIGS. 17A and 17B, the total radiant efficacy of the SAFA-IL-2m2-administered group was higher than that of the positive control group (efavaleukin-alfa-CD750) at the foot site where the arthritis was induced, confirming that the SAFA-IL-2m2 was distributed by targeting the inflammation-induced arthritis site. Also, when comparing the total radiant efficacy with the positive control group (efavaleukin-alfa-CD750), the measured values in the liver, lung, and spleen was relatively low, whereas the measured values in the kidney was high among the extracted tissues in the SAFA-IL-2m2-CF750 administered group, suggesting that the SAFA-IL-2m2-CF750 accumulated less in tissues other than the inflamed target sites compared to the positive control group (efavaleukin-alfa-CF750).

### Example 8. Validation of Efficacy of SAFA-IL-2m2 Using MRL Lupus Mouse Model

The efficacy of the drug against lupus was validated when the SAFA-IL-2m2 was administered to the MRL/lpr mouse model, which is widely used as a model for evaluating the therapeutic effect of systemic lupus erythematosus (SLE). Specifically, to the normal mouse model ((C57BL/6) and the lupus mouse model using MRL/lpr mice, mycophenolate mofetil 60 mg/kg as a positive control substance and SAFA-IL-2m2 400 μg/kg were administered along with vehicle as a negative control substance from 9 weeks of age. After the drug administration was started, proteinuria scores and lymphadenopathy scores were measured. Also, to confirm the DAI through quantitative analysis of autoantibodies in mouse blood, anti-dsDNA IgG was examined in blood obtained through heart blood collection after the experiment was completed.

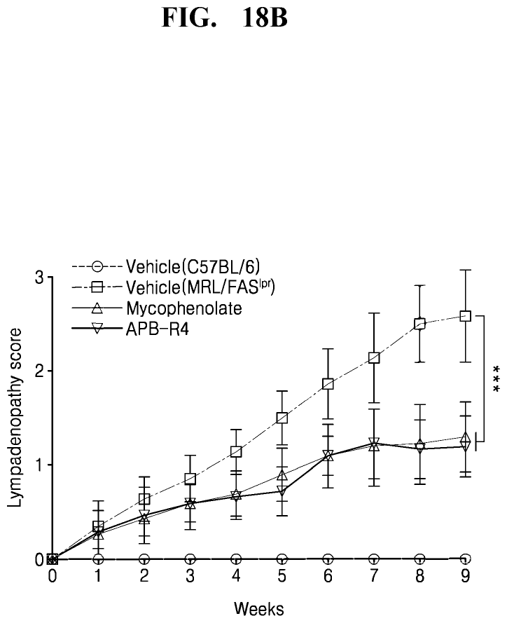

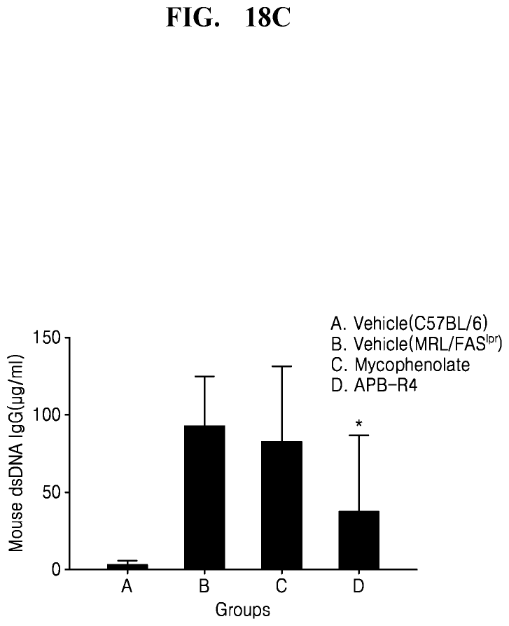

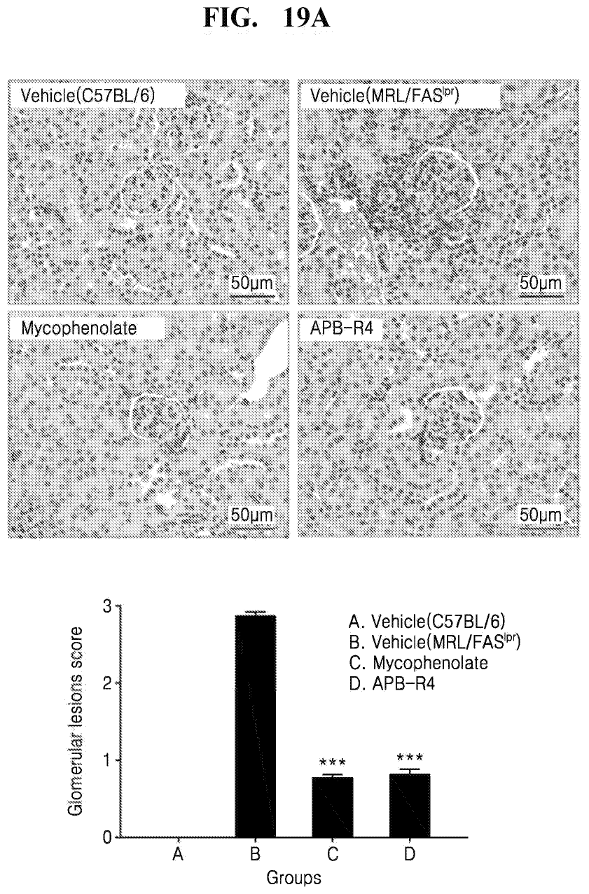

As shown in FIGS. 18A-18C, it was confirmed that the proteinuria scores and lymphadenopathy scores were statistically significantly reduced in the positive control material (mycophenolate) and the SAFA-IL-2m2-administered group compared to the negative control group (vehicle, MRL/lpr). That is, the therapeutic effect on lupus was validated to be similar to or better than mycophenolate mofetil, which is used as a standard treatment in clinical practice. Also in the results of measuring the anti-dsDNA IgG concentration, such a decreasing tendency was observed in the positive control material (mycophenolate) and the SAFA-IL-2m2-administered group compared to the negative control group (vehicle, MRL/lpr), particularly in the SAFA-IL-2m2-administered group showing a statistically significant decrease. Referring to the results above, it was validated that the SAFA-IL-2m2 effectively suppressed the production of autoantibodies and showed a superior therapeutic effect compared to mycophenolate mofetil which is a standard treatment used clinically.

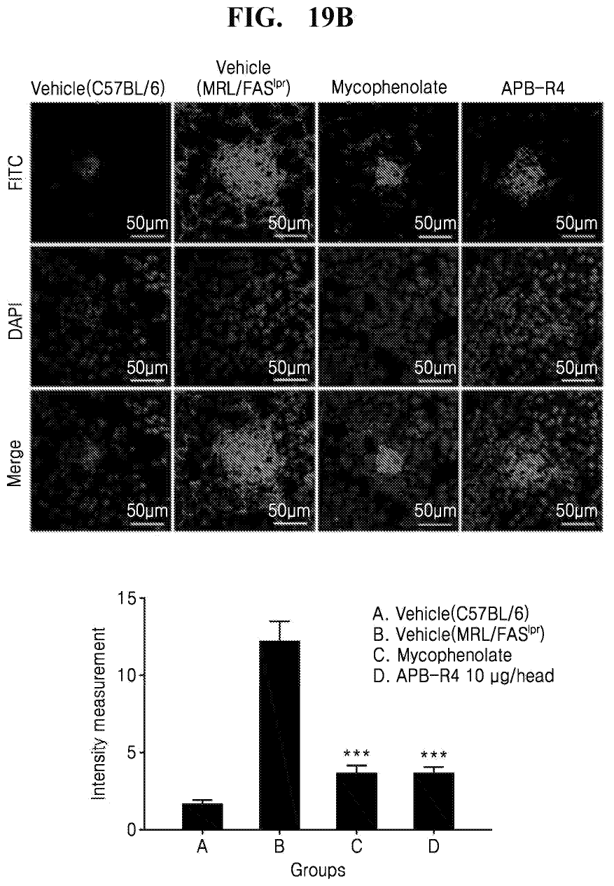

Also, after the experiment was finished, the left kidney tissue was extracted and subjected to hematoxylin and eosin (H&E) staining, and the right kidney tissue was extracted and subjected to immunofluorescence staining for IgG. As shown in FIGS. 19A and 19B, it was confirmed through the H&E staining that the administration of the SAFA-IL-2m2 not only suppressed the mesangial proliferation, but also significantly reduced inflammatory cell infiltration in glomeruli. As a result of the immunofluorescence staining for IgG, it was confirmed that the SAFA-IL-2m2 exhibited a therapeutic effect on glomerulonephritis by reducing renal infiltration of immune complexes. Based on the results above, the therapeutic effect of the SAFA-IL-2m2 on lupus was confirmed, as well as the efficacy consistent with the reduction in the frequency of moderate to severe proteinuria and the improvement of glomerulonephritis.

According to the one or more embodiments, a recombinant fusion protein is a fusion of an IL-2 mutein and an anti-serum albumin antibody and has an advantage of a relatively long dosing cycle due to an increased in vivo half-life. In addition, since side effects do not occur in the body due to low immunogenicity, the recombinant fusion protein can be effectively used in the treatment of various immune diseases including inflammatory bowel disease or systemic lupus erythematosus.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details can be made therein without departing from the spirit and scope of the disclosure as defined by the following claims.

All of the various aspects, embodiments, and options described herein can be combined in any and all variations. All publications, patents, and patent applications mentioned in this specification are herein incorporated by reference to the same extent as if each individual publication, patent, or patent application was specifically and individually indicated to be herein incorporated by reference.

What is claimed is:

1. A recombinant fusion protein comprising an interleukin-2 mutein (IL-2m) and an antigen binding fragment (Fab) that binds to serum albumin.

2. The recombinant fusion protein of claim 1, wherein the IL-2m and the Fab to serum albumin are linked to each other by a linker.

3. The recombinant fusion protein of claim 1, wherein a C-terminal region of a heavy chain of the Fab to serum albumin and the IL-2m are linked to each other by a linker.

4. The recombinant fusion protein of claim 1, wherein the IL-2m, a linker, a heavy chain comprising a heavy chain region of the Fab to serum albumin, and a light chain comprising a light chain region of the Fab to serum albumin are linked by non-covalent bonds.

5. The recombinant fusion protein of any one of claims 1-3, wherein the linker comprises 1 to 50 amino acids.

6. The recombinant fusion protein of claim 5, wherein the linker comprises an amino acid sequence of any one of SEQ ID NOS: 16, 70-85, and 88.

7. The recombinant fusion protein of claim 5, wherein the linker has an amino acid sequence of SEQ ID NO:84.

8. The recombinant fusion protein of claim 7, wherein the IL-2m, the linker, and the heavy chain comprises an amino acid sequence of SEQ ID NO:4.

9. The recombinant fusion protein of claim 1, wherein the recombinant fusion protein comprises amino acid sequences of SEQ ID NOS: 6 and 2.

10. The recombinant fusion protein of any one of claims 1-9, wherein the IL-2m comprises an amino acid sequence having at least 90% identity to SEQ ID NO:2.

11. The recombinant fusion protein of any one of claims 1-9, wherein IL-2m comprises an amino acid sequence of SEQ ID NO:2.

12. The recombinant fusion protein of any one of claims 1-9, wherein the IL-2m has an amino acid sequence of SEQ ID NO:1.

13. The recombinant fusion protein of any one of claims 1-12, wherein the Fab comprises
a heavy chain comprising a heavy chain variable domain comprising
(a) a heavy chain complementarity determining domain 1 (CDR1) comprising the amino acid sequence of SYGIS (SEQ ID NO:22),
a heavy chain complementarity determining domain 2 (CDR2) comprising the amino acid sequence of WINTYSGGTKYAQKFQG (SEQ ID NO:23), and
a heavy chain complementarity determining domain 3 (CDR3) comprising the amino acid sequence of LGHCQRGICSDALDT (SEQ ID NO:24);
(b) a heavy chain CDR1 comprising the amino acid sequence of SYGIS (SEQ ID NO:22),
a heavy chain CDR2 comprising the amino acid sequence of RINTYNGNTGYAQRLQG (SEQ ID NO:25), and
a heavy chain CDR3 comprising the amino acid sequence of LGHCQRGICSDALDT (SEQ ID NO:24);
(c) a heavy chain CDR1 comprising the amino acid sequence of NYGIH (SEQ ID NO:26),
a heavy chain CDR2 comprising the amino acid sequence of SISYDGSNKYYADSVKG (SEQ ID NO:27), and
a heavy chain CDR3 comprising the amino acid sequence of DVHYYGSGSYYNAFDI (SEQ ID NO:28);
(d) a heavy chain CDR1 comprising the amino acid sequence of SYAMS (SEQ ID NO:29),
a heavy chain CDR2 comprising the amino acid sequence of VISHDGGFQYYADSVKG (SEQ ID NO:30), and
a heavy chain CDR3 comprising the amino acid sequence of AGWLRQYGMDV (SEQ ID NO:31);
(e) a heavy chain CDR1 comprising the amino acid sequence of AYWIA (SEQ ID NO:32),
a heavy chain CDR2 comprising the amino acid sequence of MIWPPDADARYSPSFQG (SEQ ID NO:33), and
a heavy chain CDR3 comprising the amino acid sequence of LYSGSYSP (SEQ ID NO: 34); or
(f) a heavy chain CDR1 comprising the amino acid sequence of AYSMN (SEQ ID NO:35),
a heavy chain CDR2 comprising the amino acid sequence of SISSSGRYIHYADSVKG (SEQ ID NO:36), and
a heavy chain CDR3 comprising the amino acid sequence of ETVMAGKALDY (SEQ ID NO:37); and
a light chain comprising a light chain variable domain comprising
(g) a light chain CDR1 comprising the amino acid sequence of RASQSISRYLN (SEQ ID NO:38),
a light chain CDR2 comprising the amino acid sequence of GASRLES (SEQ ID NO: 39), and
a light chain CDR3 comprising the amino acid sequence of QQSDSVPVT (SEQ ID NO:40);
(h) a light chain CDR1 comprising the amino acid sequence of RASQSISSYLN (SEQ ID NO:41),
a light chain CDR2 comprising the amino acid sequence of AASSLQS (SEQ ID NO: 42), and
a light chain CDR3 comprising the amino acid sequence of QQSYSTPPYT (SEQ ID NO:43);
(i) a light chain CDR1 comprising the amino acid sequence of RASQSIFNYVA (SEQ ID NO:44),
a light chain CDR2 comprising the amino acid sequence of DASNRAT (SEQ ID NO: 45), and
a light chain CDR3 comprising the amino acid sequence of QQRSKWPPTWT (SEQ ID NO:46);
(j) a light chain CDR1 comprising the amino acid sequence of RASETVSSRQLA (SEQ ID NO:47),
a light chain CDR2 comprising the amino acid sequence of GASSRAT (SEQ ID NO: 48), and
a light chain CDR3 comprising the amino acid sequence of QQYGSSPRT (SEQ ID NO:49);
(k) a light chain CDR1 comprising the amino acid sequence of RASQSVSSSSLA (SEQ ID NO:50),
a light chain CDR2 comprising the amino acid sequence of GASSRAT (SEQ ID NO: 48), and
a light chain CDR3 comprising the amino acid sequence of QKYSSYPLT (SEQ ID NO: 51); or
(l) a light chain CDR1 comprising the amino acid sequence of RASQSVGSNLA (SEQ ID NO:52),
a light chain CDR2 comprising the amino acid sequence of GASTGAT (SEQ ID NO: 53), and
a light chain CDR3 comprising the amino acid sequence of QQYYSFLAKT (SEQ ID NO:54).

14. The recombinant fusion protein of any one of claims 1-13,
wherein the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and
wherein the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

15. The recombinant fusion protein of any one of claims 1-14, wherein the heavy chain variable domain comprises an amino acid sequence having at least 90% identity to SEQ ID NO: 55, 56, 57, 58, 59, or 60.

16. The recombinant fusion protein of any one of claims 1-15, wherein the heavy chain variable domain comprises the amino acid sequence of SEQ ID NO:60.

17. The recombinant fusion protein of any one of claims 1-16, wherein the light chain variable domain comprises an amino acid sequence having at least 90% identity to SEQ ID NO: 61, 62, 63, 64, 65, 66, or 67.

18. The recombinant fusion protein of any one of claims 1-17 wherein the light chain variable domain comprises the amino acid sequence of SEQ ID NO:67.

19. The recombinant fusion protein of any one of claims 1-14, wherein the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67.

20. The recombinant fusion protein of any one of claims 1-18, wherein the heavy chain constant domain comprises an amino acid sequence having at least 90% identity to SEQ ID NO: 68.

21. The recombinant fusion protein of any one of claims 1-18, wherein the light chain constant domain comprises an amino acid sequence having at least 90% identity to SEQ ID NO: 69 or 91.

22. The recombinant fusion protein of any one of claims 1-20, wherein the Fab comprises a heavy chain comprising the amino acid sequence of SEQ ID NO:6 and a light chain comprising the amino acid sequence of SEQ ID NO:69 or 91.

23. The recombinant fusion protein of any one of claims 1-22, wherein the Fab comprises a heavy chain constant 1 domain comprising the amino acid sequence of SEQ ID NO: 68 linked to the heavy chain variable domain; and a light chain constant domain comprising the amino acid sequence of SEQ ID NO:69 or 91 linked to the light chain variable domain.

24. A nucleic acid encoding the recombinant fusion protein of any one of claims 1-23.

25. An expression vector comprising the nucleic acid of claim 24.

26. A cell transformed with the expression vector of claim 25.

27. A composition comprising the recombinant fusion protein of any one of claims 1-23 and a carrier.

28. A pharmaceutical composition comprising the recombinant fusion protein of any one of claims 1-23 and a pharmaceutically acceptable carrier.

29. A kit comprising the composition of claim 27 or 28 and a label comprising instructions for a use.

30. A method of treating for preventing or treating autoimmune disease or inflammatory neurological disease in a subject in need thereof, comprising administering the pharmaceutical composition of claim 28 to the subject.

31. The method of claim 30, wherein the autoimmune disease is selected from the group consisting of Crohn's disease, ulcerative colitis, spondylitis ankylopoietica, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, restenosis of a blood vessel, glomerulonephritis, and gastrointestinal allergy, and the inflammatory neurological disease is selected from the group consisting of amyotrophic lateral sclerosis and Parkinson's disease.

32. The use of a pharmaceutical composition for preventing or treating autoimmune disease or inflammatory neurological disease, comprising the recombinant fusion protein of claim 1 as an active ingredient.

33. The use of claim 32, wherein the autoimmune disease is inflammatory bowel disease.

34. The use of claim 32, wherein the autoimmune disease is selected from the group consisting of Crohn's disease, ulcerative colitis, spondylitis ankylopoietica, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, restenosis of a blood vessel, glomerulonephritis, and gastrointestinal allergy, and the inflammatory neurological disease is selected from the group consisting of amyotrophic lateral sclerosis and Parkinson's disease.
